(19) United States
(12) Patent Application Publication
(10) Pub. No.: US 2023/0210947 A1
(43) Pub. Date: Jul. 6, 2023

(54) TRANSFORMING GROWTH FACTOR-BETA LIGAND TRAPS FOR THE TREATMENT OF DISEASE

(71) Applicant: BRISTOL-MYERS SQUIBB COMPANY, Princeton, NJ (US)

(72) Inventors: Raymond Perez, Princeton, NJ (US); Bruce S. Fischer, East Brunswick, NJ (US); Lora Hamuro, Lawrenceville, NJ (US)

(21) Appl. No.: 17/847,927

(22) Filed: Jun. 23, 2022

Related U.S. Application Data
(60) Provisional application No. 63/214,585, filed on Jun. 24, 2021.
(60) Provisional application No. 63/214,588, filed on Jun. 24, 2021.

(51) Int. Cl. A61K 38/17; A61K 39/395; C07K 16/28; A61P 35/00

(52) U.S. Cl. CPC A61K 38/179; A61K 39/3955; A61P 35/00; C07K 16/2818; A61K 2039/545

(57) ABSTRACT

The present application relates to methods using Transforming Growth Factor-β (TGF-β) ligand traps. The TGF-β ligand traps described herein may be suitable for combination therapy with an immunotherapy, for treating a disease or disorder such as a cancer. The TGF-β ligand traps described herein may also be suitable for monotherapy for treating a disease or disorder such as a cancer. In particular, provided herein are methods and compositions for treating a disease or disorder such as a cancer by administering a TGF-β ligand trap in combination with an immune checkpoint inhibitor.

### PRIORITY

This application claims the benefit of priority to U.S. Serial No. 63/214,585 filed Jun. 24, 2021 and U.S. Serial No. 63/214,588 filed Jun. 24, 2021, each of which is incorporated herein by reference in its entirety.

### SEQUENCE LISTING

This application incorporates by reference a Sequence Listing submitted with this application as text file entitled “14247-669-999_Sequence_Listing.txt” created on Jun. 23, 2022 and having a size of 32,722 bytes.

### FIELD

The present invention relates to methods of using a Transforming Growth Factor-β (TGF-β) ligand trap, including combinations of a TGF-β ligand trap and an immunotherapy, for treating a disease or disorder such as a cancer.

### 1. Background

Cancer can induce significant suppression of the immune system and escape from the immune surveillance mechanisms of the host. Dysregulation of host immune system is now considered one important hallmark of cancer (Hanahan et al., Cell, 2011, 144, 646-674). The interactions between cancer and the host immune system have been extensively studied and many types of immunotherapies have been explored for cancer treatment. One class of immunotherapy is agents targeting specific immune checkpoint proteins that play critical roles in regulating T cell activation and proliferation. These proteins function as co-receptors on the surfaces of T cells and help regulate T cell responses following T cell activation (Wolchok et al., Cancer J., 2010, 16, 311-317). The two best characterized checkpoint proteins are cytotoxic T-lymphocyte antigen 4 (CTLA-4) and programmed death-1 (PD-1), both serve as negative regulators of T cell activation.

While immunotherapy with checkpoint inhibitors have changed survival expectations for many cancer patients, not all patients respond to them and some stop responding over time (Jenkins et al., Br. J. Cancer, 2018, 118(1), 9-16). In investigating the mechanisms of resistance to immune checkpoint inhibitors, it has been found that tumors can evolve to evade both innate and adaptive arms of the immune system, thereby rendering immunotherapy with immune checkpoint inhibitors ineffective (Pitt et al., Immunity, 2016, 44(6), 1255-1269; Restifo et al., Nat. Rev. Cancer, 2016, 12(2), 121-126). There still remains a need of effective combination therapies to overcome both innate and acquired resistance to immunotherapy with immune checkpoint inhibitors.

TGF-P ligand traps (e.g., M7824 (Knudson etal., Oncoimmunology 7(5):e1426519 (2018)) or AVID200 (Thwaites et al., Blood 130:2532 (2017))) are comprised of isolated TGF-β receptors that inhibit the binding of TGF-β ligand to its cognate receptor on cells. TGF-β ligand traps prevent TGF ligands from binding to TGF receptors, thereby preventing TGF-β-mediated signaling. The TGF-β ligand can exist in three known isoforms, TGF-β1, TGF-β2, and TGF-β3. TGF-β1 and TGF-β3 have been identified as oncogenic isoforms while TGF-β2 promotes normal cardiac function. Additionally, the role of TGF-β in metastasis has been described in O’Connor-McCourt et al., Cancer Research, 2018, 1759. Therefore, in the design of TGF-β ligand traps for use as cancer therapeutics, it is beneficial for the ligand trap to bind and sequester TGF-β isoforms TGF-β1 and TGF-β3 specifically while not binding to TGF-β2. TGF-β ligand traps have been promising new immunotherapy that enhances desirable anti-tumor immunity.

### 2. Summary

Provided herein are methods of treating cancer in a subject in need thereof comprising administering to the subject a polypeptide comprising an amino acid sequence selected from any one of SEQ ID NO:1 to 5 at a dose of about 400 mg to about 1600 mg. Provided herein are methods of treating cancer in a subject in need thereof comprising a polypeptide comprising an amino acid sequence that is at least 80-99%, for example 85%, 86%, 87%, 88%, 89%, 90%, 91%, 92%, 93%, 94%, 95%, 96%, 97%, 98%, or 99%, identical to any one of the amino acid sequences of SEQ ID NO:1, SEQ ID NO: 2, SEQ ID NO: 3, SEQ ID NO: 4, and SEQ ID NO: 5 at a dose of about 400 mg to about 1600 mg. In various embodiments, the dose used in the method is the therapeutically effective amount.

In various embodiments, the method further comprises administering about 360 mg Q3W of nivolumab to the subject.

In various embodiments, the method further comprises administering about 480 mg Q4W of nivolumab to the subject.

In various embodiments, the treatment with a polypeptide comprising an amino acid sequence selected from any one of SEQ ID NO:1 to 5 comprises administering the polypeptide to the subject a single time per dosing cycle.

In various embodiments, the polypeptide is administered using a schedule, regimen and/or dosing cycle. In various embodiments, the schedule, regimen and/or dosing cycle begins on day 1 and ends on day 21.

In various embodiments, the schedule, regimen and/or dosing cycle is repeated about 10 to about 20 times. In various embodiments, the schedule, regimen and/or dosing cycle is repeated about 15 to about 25 times. In various embodiments, the schedule, regimen and/or dosing cycle is repeated about 20 to about 30 times. In various embodiments, the schedule, regimen and/or dosing cycle is repeated about 25 to about 35 times. In various embodiments, the schedule, regimen and/or dosing cycle is repeated about 30 to about 40 times. In various embodiments, the schedule, regimen and/or dosing cycle is repeated about 35 times.

In various embodiments, the dose of the polypeptide comprising an amino acid sequence selected from any one of SEQ ID NO: 1 to 5 is about 400 mg. In various embodiments, the dose of the polypeptide comprising an amino acid sequence selected from any one of SEQ ID NO: 1 to 5 is about 800 mg. In various embodiments, the dose of the polypeptide comprising an amino acid sequence selected from any one of SEQ ID NO:1 to 5 is about 1200 mg. In various embodiments, the dose of the polypeptide comprising an amino acid sequence selected from any one of SEQ ID NO: 1 to 5 is about 1600 mg.

In various embodiments, the dose of the polypeptide comprising an amino acid sequence selected from any one of SEQ ID NO: 1 to 5 is 400 mg. In various embodiments, the dose of the polypeptide comprising an amino acid sequence selected from any one of SEQ ID NO:1 to 5 is 800 mg. In various embodiments, the dose of the polypeptide comprising an amino acid sequence selected from any one of SEQ ID NO: 1 to 5 is 1200 mg. In various embodiments, the dose of the polypeptide comprising an amino acid sequence selected from any one of SEQ ID NO: 1 to 5 is 1600 mg.

In various embodiments, the polypeptide is administered Q3W. In various embodiments, the polypeptide is administered Q3W at a dose of 400 mg. In various embodiments, the polypeptide is administered Q3W at a dose of 800 mg. In various embodiments, the polypeptide is administered Q3W at a dose of 1200 mg. In various embodiments, the polypeptide is administered Q3W at a dose of 1600 mg. In various embodiments, the polypeptide comprises the amino acid sequence of any one of SEQ ID Nos: 1-5. In various embodiments, the polypeptide comprises the amino acid sequence of SEQ ID NO: 3.

In various embodiments, the polypeptide is administered Q4W. In various embodiments, the polypeptide is administered Q4W at a dose of 400 mg. In various embodiments, the polypeptide is administered Q4W at a dose of 800 mg. In various embodiments, the polypeptide is administered Q4W at a dose of 1200 mg. In various embodiments, the polypeptide is administered Q4W at a dose of 1600 mg. In various embodiments, the polypeptide comprises the amino acid sequence of any one of SEQ ID Nos: 1-5. In various embodiments, the polypeptide comprises the amino acid sequence of SEQ ID NO: 3.

In various embodiments, the nivolumab and the polypeptide comprising an amino acid sequence selected from any one of SEQ ID NO:1 to 5 are formulated separately. In various embodiments, the nivolumab and the polypeptide comprising an amino acid sequence selected from any one of SEQ ID NO: 1 to 5 are formulated together in a single pharmaceutical composition that further comprises an excipient.

In various embodiments, the polypeptide comprises an amino acid sequence consisting of SEQ ID 1. In various embodiments, the polypeptide comprises an amino acid sequence consisting of SEQ ID 2. In various embodiments, the polypeptide comprises an amino acid sequence consisting of SEQ ID 3. In various embodiments, the polypeptide comprises an amino acid sequence consisting of SEQ ID 4. In various embodiments, the polypeptide comprises an amino acid sequence consisting of SEQ ID 5.

In various embodiments, the cancer comprises an advanced solid tumor.

In various embodiments, the cancer is selected from the group consisting of non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC), renal cell carcinoma (RCC), and pancreatic ductal adenocarcinoma (PDAC).

In various embodiments, the method results in a reduction in the size and/or growth of a tumor or in the number of tumors associated with the cancer.

In various embodiments, the subject is resistant or refractory to or intolerant of existing standard cancer therapies known to provide clinical benefit.

In various embodiments, the subject is resistant or refractory to immunotherapy that inhibits PD-1 signaling.

In various embodiments, the subject is resistant or refractory to anti-PD-(L)1-based immunotherapy.

Provided herein is use of the polypeptide comprising an amino acid sequence selected from any one of SEQ ID NO: 1 to 5 alone and/or in combination with nivolumab described above for use in therapy in treatment of cancer.

Provided herein are methods of treating cancer in a subject in need thereof, wherein the method comprises administering a nivolumab treatment and a treatment with TGFβ trap. In various embodiments, the treatment with TGFβ trap comprises a polypeptide comprising an amino acid sequence selected from any one of SEQ ID NO: 1 to 5, or a fragment thereof, or an amino acid sequence that is at least 80-99%, for example 85%, 86%, 87%, 88%, 89%, 90%, 91%, 92%, 93%, 94%, 95%, 96%, 97%, 98% or 99%, identical to any one of the amino acid sequences of SEQ ID NO:1, SEQ ID NO: 2, SEQ ID NO: 3, SEQ ID NO: 4, SEQ ID NO: 5. In various embodiments,
(i) the nivolumab treatment comprises administering about 360 mg to about 480 mg of nivolumab to the subject on about day 1 of a dosing cycle; and
(ii) the treatment with a polypeptide comprising an amino acid sequence selected from any one of SEQ ID NO: 1 to 5 comprises administering a dose of about 400 mg to about 1600 mg of the polypeptide to the subject on about day 1 of the dosing cycle. SEQ ID NO: and SEQ ID: are used interchangeably herein.

In certain embodiments, the nivolumab treatment comprises administering about 360 mg of nivolumab to the subject. In certain embodiments, the nivolumab treatment consists of administering about 360 mg of nivolumab to the subject. In certain embodiments, the nivolumab treatment comprises administering 360 mg of nivolumab to the subject. In certain embodiments, the nivolumab treatment consists of administering 360 mg of nivolumab to the subject.

In certain embodiments, the nivolumab treatment comprises administering nivolumab to the subject a single time per dosing cycle. In certain embodiments, the nivolumab treatment consists of administering nivolumab to the subject a single time per dosing cycle. In certain embodiments, nivolumab is administered once on day 1 of the dosing cycle.

In certain embodiments, nivolumab is administered once every 2 weeks. In certain embodiments, nivolumab is administered once every 4 weeks.

In certain embodiments, the TGF-β ligand trap (e.g., AVID200) is administered once every 1, 2, 3, or 4 weeks. In a specific embodiment, the TGF-β ligand trap (e.g., AVID200) is administered once every week. In a specific embodiment, the TGF-β ligand trap (e.g., AVID200) is administered once every 2 weeks. In another specific embodiment, the TGF-β ligand trap (e.g., AVID200) is administered once every 3 weeks. In another specific embodiment, the TGF-β ligand trap (e.g., AVID200) is administered once every 4 weeks.

In certain embodiments, the treatment with a polypeptide comprising an amino acid sequence selected from any one of SEQ ID NO:1 to 5 comprises administering the polypeptide to the subject a single time per dosing cycle. In certain embodiments, the treatment with a polypeptide comprising an amino acid sequence selected from any one of SEQ ID NO:1 to 5 consists of administering the polypeptide to the subject a single time per dosing cycle. In certain embodiments, the polypeptide is administered once on day 1 of the dosing cycle.

In certain embodiments, the dosing cycle begins on day 1 and ends on day 21.

In certain embodiments, the dosing cycle is repeated about 10 to about 20 times.

In certain embodiments, the dosing cycle is repeated about 15 to about 25 times.

In certain embodiments, the dosing cycle is repeated about 20 to about 30 times.

In certain embodiments, the dosing cycle is repeated about 25 to about 35 times.

In certain embodiments, the dosing cycle is repeated about 30 to about 40 times.

In certain embodiments, the dosing cycle is repeated about 35 times.

Also provided herein are methods of treating cancer in a subject in need thereof, wherein the method comprises administering a nivolumab treatment and a treatment with a polypeptide comprising an amino acid sequence selected from any one of SEQ ID NO:1 to 5, or a fragment thereof, or an amino acid sequence that is at least 80%-99%, for example 85%, 86%, 87%, 88%, 89%, 90%, 91%, 92%, 93%, 94%, 95%, 96%, 97%, 98% or 99%, identical to any one of the amino acid sequences of SEQ ID NO: 1, SEQ ID NO: 2, SEQ ID NO: 3, SEQ ID NO: 4, SEQ ID NO: 5, or a fragment thereof; wherein
(i) the nivolumab treatment comprises administering about 360 mg to about 480 mg of nivolumab to the subject on about day 1 of a dosing cycle; and
(ii) the treatment with a polypeptide comprising an amino acid sequence selected from any one of SEQ ID NO: 1 to 5 comprises administering a dose of about 400 mg to about 1600 mg of the polypeptide to the subject on about day 1 of the dosing cycle and a dose of about 400-1600 mg of the polypeptide on about day 15 of the dosing cycle.

In certain embodiments, the dose of the polypeptide that is administered on about day 1 and the dose of the polypeptide that is administered on about day 15 are the same. In certain embodiments, the dose of the polypeptide that is administered on day 1 and the dose of the polypeptide that is administered on day 15 are the same.

In certain embodiments, the nivolumab treatment comprises administering about 480 mg of nivolumab to the subject. In certain embodiments, the nivolumab treatment consists of administering about 480 mg of nivolumab to the subject. In certain embodiments, the nivolumab treatment comprises administering 480 mg of nivolumab to the subject. In certain embodiments, the nivolumab treatment consists of administering 480 mg of nivolumab to the subject.

In certain embodiments, the nivolumab treatment comprises administering nivolumab to the subject a single time per dosing cycle. In certain embodiments, the nivolumab treatment consists of administering nivolumab to the subject once per dosing cycle. In certain embodiments, nivolumab is administered once on day 1 of the dosing cycle.

In certain embodiments, the treatment with a polypeptide comprises administering the amino acid sequence selected from any one of SEQ ID NO: 1 to 5 to the subject twice per dosing cycle. In certain embodiments, the treatment with a polypeptide comprises administering the amino acid sequence selected from any one of SEQ ID NO: 1 to 5 to the subject two times per dosing cycle. In certain embodiments, the treatment with a polypeptide consists of administering the amino acid sequence selected from any one of SEQ ID NO:1 to 5 twice per dosing cycle. In certain embodiments, the polypeptide is administered on day 1 and on day 15 of the dosing cycle.

In certain embodiments, the dosing cycle begins on day 1 and ends on day 28.

In certain embodimets, the dosing cycle is repeated about 10 to about 20 times.

In certai embodiments, the dosing cycle is repeated about 15 to about 25 times.

In certain embodiments, the dosing cycle is repeated about 20 to about 30 times.

In certain embodiments, the dosing cycle is repeated about 25 to about 35 times.

In certain embodiments, the dosing cycle is repeated about 30 to about 40 times.

In certain embodiments, wherein the dosing cycle is repeated about 26 times.

In certain embodiments of any of the methods provided herein, the dose of the polypeptide comprising an amino acid sequence selected from any one of SEQ ID NO:1 to 5 is about 400 mg. In certain embodiments, the dose is 400 mg.

In certain embodiments of any of the methods provided herein, the dose of the polypeptide comprising an amino acid sequence selected from any one of SEQ ID NO:1 to 5 is about 800 mg. In certain embodiments, the dose is 800 mg.

In certain embodiments of any of the methods provided herein, the dose of the polypeptide comprising an amino acid sequence selected from any one of SEQ ID NO:1 to 5 is about 1200 mg. In certain embodiments, the dose is 1200 mg.

In certain embodiments of any of the methods provided herein, the dose of the polypeptide comprising an amino acid sequence selected from any one of SEQ ID NO:1 to 5 is about 1600 mg. In certain embodiments, the dose is 1600 mg.

In certain embodiments of any of the methods provided herein, the nivolumab is present in a pharmaceutical composition that further comprises an excipient.

In certain embodiments of any of the methods provided herein, the polypeptide comprising an amino acid sequence selected from any one of SEQ ID NO: 1 to 5 is present in a pharmaceutical composition that further comprises an excipient. In various embodiments, the TGF-β ligand trap is formulated as a solution in a single-use vial. In various embodiments, the unit dose strength of the TGF-β ligand trap polypeptide in the formulation is 80 mg (8 mg/mL). In various embodiments, the nivolumab is formulated as a solution in a single use vial. In various embodiments, the unit dose strength of the nivolumab is 100 mg (10 mg/mL).

In certain embodiments of any of the methods provided herein, the nivolumab and the polypeptide comprising an amino acid sequence selected from any one of SEQ ID NO:1 to 5 are formulated together in a single pharmaceutical composition. In various embodiments, the single pharmaceutical composition further comprises an excipient.

In certain embodiments of any of the methods provided herein, the TGF-β ligand trap is a polypeptide comprising an amino acid sequence that is at least 80%-85%, 85%-90%, 90%-95%, or 95%-99% identical to any one of the amino acid sequences of SEQ ID NO: 1, SEQ ID NO: 2, SEQ ID NO: 3, SEQ ID NO: 4, SEQ ID NO: 5, or a fragment thereof.

In certain embodiments of any of the methods provided herein, the TGF-β ligand trap comprises a polypeptide comprising the amino acid sequence of SEQ ID NO: 1 or a fragment thereof. In certain embodiments, the TGF-β ligand trap is a polypeptide comprising the amino acid sequence of SEQ ID NO: 1 or a fragment thereof. In certain embodiments, the TGF-β ligand trap is a polypeptide consisting of the amino acid sequence of SEQ ID NO: 1 or a fragment thereof. In certain embodiments, the polypeptide comprises an amino acid sequence consisting of SEQ ID 1.

In certain embodiments of any of the methods provided herein, the TGF-β ligand trap comprises a polypeptide comprising the amino acid sequence of SEQ ID NO: 2 or a fragment thereof. In certain embodiments, the TGF-β ligand trap is a polypeptide comprising the amino acid sequence of SEQ ID NO: 2 or a fragment thereof. In certain embodiments, the TGF-β ligand trap is a polypeptide consisting of the amino acid sequence of SEQ ID NO: 2 or a fragment thereof. In certain embodiments, the polypeptide comprises an amino acid sequence consisting of SEQ ID 2.

In certain embodiments of any of the methods provided herein, the TGF-β ligand trap comprises a polypeptide comprising the amino acid sequence of SEQ ID NO: 3 or a fragment thereof. In certain embodiments, the TGF-β ligand trap is a polypeptide comprising the amino acid sequence of SEQ ID NO: 3 or a fragment thereof. In certain embodiments, the TGF-β ligand trap is a polypeptide consisting of the amino acid sequence of SEQ ID NO: 3 or a fragment thereof. In certain embodiments, the polypeptide comprises an amino acid sequence consisting of SEQ ID 3.

In certain embodiments of any of the methods provided herein, the TGF-β ligand trap comprises a polypeptide comprising the amino acid sequence of SEQ ID NO: 4 or a fragment thereof. In certain embodiments, the TGF-β ligand trap is a polypeptide comprising the amino acid sequence of SEQ ID NO: 4 or a fragment thereof. In certain embodiments, the TGF-β ligand trap is a polypeptide consisting of the amino acid sequence of SEQ ID NO: 4 or a fragment thereof. In certain embodiments, the polypeptide comprises an amino acid sequence consisting of SEQ ID 4.

In certain embodiments of any of the methods provided herein, the TGF-β ligand trap comprises a polypeptide comprising the amino acid sequence of SEQ ID NO: 5 or a fragment thereof. In certain embodiments, the TGF-β ligand trap is a polypeptide comprising the amino acid sequence of SEQ ID NO: 5 or a fragment thereof. In certain embodiments, the TGF-β ligand trap is a polypeptide consisting of the amino acid sequence of SEQ ID NO: 5 or a fragment thereof. In certain embodiments, the polypeptide comprises an amino acid sequence consisting of SEQ ID 5.

In certain embodiments of any of the methods provided herein, the cancer comprises a solid tumor, for example, an advanced solid tumor.

In certain embodiments of any of the methods provided herein, wherein the cancer is selected from the group consisting of non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC) and pancreatic ductal adenocarcinoma (PDAC).

In certain embodiments of any of the methods provided herein, the method results in a reduction in the size of a tumor or in the number of tumors associated with the cancer.

In certain embodiments of any of the methods provided herein, the subject is resistant or refractory to or intolerant of existing standard cancer therapies known to provide clinical benefit.

In certain embodiments of any of the methods provided herein, the subject is resistant or refractory to immunotherapy that inhibits PD-1 signaling.

In certain embodiments of any of the methods provided herein, the subject is resistant or refractory to anti-PD-(L)1-based immunotherapy.

Also provided herein are methods of preventing or treating a cancer in a subject comprising administering to a subject in need thereof an effective amount of a TGF-β ligand trap and nivolumab.

In certain embodiments, the cancer is resistant or refractory to anti-PD1 based immunotherapy or anti-PD-L1-based immunotherapy.

In certain embodiments, the anti-PD-1 based immunotherapy comprises an anti-PD-1 antibody.

In certain embodiments, the anti-PD-1 antibody is nivolumab.

In certain embodiments, the nivolumab is present in a pharmaceutical composition that further comprises an excipient.

In certain embodiments, the TGF-β ligand trap is present in a pharmaceutical composition that further comprises an excipient.

In certain embodiments, the nivolumab and the TGF-β ligand trap are formulated together in a single pharmaceutical composition. In various embodiments, the pharmaceutical composition further comprises an excipient.

In certain embodiments, the TGF-β ligand trap is a polypeptide comprising an amino acid sequence selected from any one of SEQ ID NO:1 to 5, or a fragment thereof, or an amino acid sequence that is at least 80%-99%, for example 85%, 86%, 87%, 88%, 89%, 90%, 91%, 92%, 93%, 94%, 95%, 96%, 97%, 98% or 99%, identical to any one of the amino acid sequences of SEQ ID NO:1, SEQ ID NO: 2, SEQ ID NO: 3, SEQ ID NO: 4, SEQ ID NO: 5. In certain embodiments of any of the methods provided herein, the TGF-β ligand trap is a polypeptide comprising an amino acid sequence that is at least 80%-85%, 85%-90%, 90%-95%, or 95%-99% identical to any one of the amino acid sequences of SEQ ID NO: 1, SEQ ID NO: 2, SEQ ID NO: 3, SEQ ID NO: 4, SEQ ID NO: 5, or a fragment thereof.

In certain embodiments, the TGF-β ligand trap comprises a polypeptide comprising the amino acid sequence of SEQ ID NO: 1 or a fragment thereof. In certain embodiments, the TGF-β ligand trap is a polypeptide comprising the amino acid sequence of SEQ ID NO: 1 or a fragment thereof. In certain embodiments, the TGF-β ligand trap is a polypeptide consisting of the amino acid sequence of SEQ ID NO: 1 or a fragment thereof. In certain embodiments, the polypeptide comprises an amino acid sequence consisting of SEQ ID 1.

In certain embodiments, the TGF-β ligand trap comprises a polypeptide comprising the amino acid sequence of SEQ ID NO: 2 or a fragment thereof. In certain embodiments, the TGF-β ligand trap is a polypeptide comprising the amino acid sequence of SEQ ID NO: 2 or a fragment thereof. In certain embodiments, the TGF-β ligand trap is a polypeptide consisting of the amino acid sequence of SEQ ID NO: 2 or a fragment thereof. In certain embodiments, the polypeptide comprises an amino acid sequence consisting of SEQ ID 2.

In certain embodiments, the TGF-β ligand trap comprises a polypeptide comprising the amino acid sequence of SEQ ID NO: 3 or a fragment thereof. In certain embodiments, the TGF-β ligand trap is a polypeptide comprising the amino acid sequence of SEQ ID NO: 3 or a fragment thereof. In certain embodiments, the TGF-β ligand trap is a polypeptide consisting of the amino acid sequence of SEQ ID NO: 3 or a fragment thereof. In certain embodiments, the polypeptide comprises an amino acid sequence consisting of SEQ ID 3.

In certain embodiments, the TGF-β ligand trap comprises a polypeptide comprising the amino acid sequence of SEQ ID NO: 4 or a fragment thereof. In certain embodiments, the TGF-β ligand trap is a polypeptide comprising the amino acid sequence of SEQ ID NO: 4 or a fragment thereof. In certain embodiments, the TGF-β ligand trap is a polypeptide consisting of the amino acid sequence of SEQ ID NO: 4 or a fragment thereof. In certain embodiments, the polypeptide comprises an amino acid sequence consisting of SEQ ID 4.

In certain embodiments, the TGF-β ligand trap comprises a polypeptide comprising the amino acid sequence of SEQ ID NO: 5 or a fragment thereof. In certain embodiments, the TGF-β ligand trap is a polypeptide comprising the amino acid sequence of SEQ ID NO: 5 or a fragment thereof. In certain embodiments, the TGF-β ligand trap is a polypeptide consisting of the amino acid sequence of SEQ ID NO: 5 or a fragment thereof. In certain embodiments, the polypeptide comprises an amino acid sequence consisting of SEQ ID 5.

In certain embodiments, the TGF-β ligand trap is AVID200.

In certain embodiments, the method comprises administering a therapeutically effective amount of the TGF-β ligand trap to the subject.

In certain embodiments, the therapeutically effective amount is about 400 mg to about 1600 mg of the TGF-β ligand trap to the subject.

In certain embodiments, the therapeutically effective amount comprises at least one dose selected from the group consisting of: about 400 mg, about 800 mg, about 1200 mg, and about 1600 mg. In certain embodiments, the therapeutically effective amount comprises at least one dose selected from the group consisting of: 400 mg, 800 mg, 1200 mg, and 1600 mg.

In certain embodiments, wherein the therapeutically effective amount is about 400 mg. In certain embodiments, the amount is 400 mg.

In certain embodiments, the therapeutically effective amount is about 800 mg. In certain embodiments, the amount is 800 mg.

In certain embodiments, the therapeutically effective amount is about 1200 mg. In certain embodiments, the amount is 1200 mg.

In certain embodiments, the therapeutically effective amount is about 1600 mg. In certain embodiments, the amount is 1600 mg.

In certain embodiments, the method comprises administering the TGF-β ligand trap every two weeks (Q2W). In certain embodiments, the TGF-β ligand trap is administered Q2W.

In certain embodiments, the method comprises administering the TGF-β ligand trap every three weeks (Q3W). In certain embodiments, the TGF-β ligand trap is administered Q3W.

In certain embodiments, the TGF-β ligand trap is administered by intravenous infusion. In various embodiments, the intravenous infusion of TGF-β ligand trap is performed over 30-90 minutes. For example, TGF-β ligand trap is administered Q2W or Q3W and is infused over 30 minutes. In certain embodiments, the TGF-β ligand trap polypeptide is administered Q2W or Q3W and is infused over 60 to 90 minutes. In various embodiments, the intravenous infusion of TGF-β ligand trap is performed over 30-90 minutes and comprises an observation period after all infusions in Cycle 1 for each participant. For example, the observation period is 60 minutes. For example, the method is a TGF-β ligand trap polypeptide monotherapy. Alternatively, the method is a combination therapy comprising the TGFβ ligand trap and a second therapeutic agent, e.g., nivolumab.

In certain embodiments, nivolumab is administered at a dose of about 360 mg. In certain embodiments, the dose is 360 mg.

In certain embodiments, nivolumab is administered at a dose of about 480 mg. In certain embodiments, the dose is 480 mg.

In certain embodiments, nivolumab is administered every three weeks Q3W.

In certain embodiments, nivolumab is administered every four weeks (Q4W).

In certain embodiments, nivolumab is administered by intravenous infusion. In various embodiments, the intravenous infusion of nivolumab is performed over 30 minutes. For example, nivolumab is administered Q3W or Q4W and is infused over 30 minutes. In certain embodiments, the TGF-β ligand trap is administered Q2W and nivolumab is administred Q3W. In certain embodiments, the TGF-β ligand trap is administered Q2W and nivolumab is administered Q4W. In certain embodiments, the TGF-β ligand trap polypeptide is administered at dosage levels of 400 mg, 800 mg, 1200 mg, or 1600 mg. In certain embodiments, the TGF-β ligand trap polypeptide (e.g., 400 mg, 800 mg, 1200 mg, or 1600 mg) is administered according to either a Q3W or Q2W dosing cycle schedule. In a specific embodiment of Q3W administration of the TGF-β ligand trap polypeptide (400 mg, 800 mg, 1200 mg, or 1600 mg), 360 mg of nivolumab is administered according to a Q3W dosing cycle schedule. In a specific embodiment of Q2W administration of the TGF-β ligand trap polypeptide (400 mg, 800 mg, 1200 mg, or 1600 mg), 480 mg of nivolumab is administered according to a Q4W dosing cycle schedule.

In some embodiments, the TGF-β ligand trap provided herein (e.g., SEQ ID NOS; 1-5 and AVID200) is administered first and nivolumab provided herein is administered second. Alternatively, the nivolumab provided herein is administered first and the TGF-β ligand trap provided herein (e.g., SEQ ID NOS; 1-5 and AVID200) is administered second.

In certain embodiments, the cancer is relapsed or refractory.

In certain embodiments, the cancer is relapsed or refractory to chemotherapy, radiation therapy, or immunotherapy.

In certain embodiments, the relapsed or refractory cancer is resistant to treatment with nivolumab.

In certain embodiments, the cancer is selected from the group consisting of NSCLC, UC, colorectal cancer, SCCHN, HCC, ovarian cancer, breast cancer, and pancreatic cancer.

In certain embodiments, the method comprising administering nivolumab and administering the TGFβ trap results in a reduction in the size of a tumor or in the number of tumors associated with the cancer.

In certain embodiments of any of the methods provided herein, the method comprising administering nivolumab and administering the TGFβ trap results in a reduction and/or modulation of at least one indicia of the presence or progression of the cancer. In certain embodiments, reduction or modulation of at least one indicia of the presence or progression of the cancer can include, but are not limited to, biomarkers such as gene expression profiling for TGF-β epithelial-mesenchymal transition/cancer-associated fibroblasts (EMT/CAF) and interferon gamma (IFNy) signatures, cluster of differentiation 8 (CD8) tumor infiltrating lymphocytes (TIL), T/NK cells, effect on TGF-β signaling pathways, cytokine profiling in tumors and/or periphery, a peptide or a protein profiling in tissues, circulating micro ribonucleic acid, circulating deoxyribonucleic acid (ctDNA), and whole exome sequencing. In an embodiment, the biomarker is a protein such as collagen, TGFβ1, and/or TGFβ3.

In certain embodiments of any of the methods provided herein, the method further comprising administering at least one therapeutic agent.

In certain embodiments, the at least one therapeutic agent comprises a therapeutic antibody, vaccine (e.g., a human papilloma virus vaccine), anti-cancer therapeutic agent or immunomodulatory drug. In various embodiments, the treatments disclosed herein (e.g., nivolumab and TGF-β ligand trap) may be used alone, or in association with radiation therapy.

In certain embodiments of any of the methods provided herein, the cancer is associated with expression of TGF-β, for example, high expression levels of TGF-β. For example, the cancer is associated with expression of TGF-β1 and/or TGF-β3.

In certain embodiments of any of the methods provided herein, the methods further include determining at least one clinical endpoint for the subject administered nivolumab and the TGF-β trap. In various embodiments, the at least one clinical endpoint includes, but is not limited to, determining the incidence of adverse events, serious adverse events, adverse events meeting dose limiting toxicity criteria, adverse events leading to discontinuation, and death; protocol-defined maximum tolerated dose or maximum administered dose. In certain embodiments, determining the at least one clinical endpoint(s) comprises measurement of a pharmacokinetic parameter of the TGF-β ligand trap, e.g., an TGF-β trap that comprises a polypeptide. In certain embodiments, determining the at least one clinical endpoint comprises determining an objective response rate and duration of response to the method. In certain embodiments, determining the at least one clinical endpoint comprises determining overall survival, progression-free survival, or progression-free survival rate associated resulting from the method. In certain embodiments, determining the at least one clinical endpoint comprises evaluating and/or detecting a change in a tumor biomarker and/or peripheral biomarker between a baseline sample (i.e., a sample from the subject prior to treatment by the method described) and a post-treatment sample. In certain embodiments, determining the at least one clinical endpoint comprises evaluating an association measure from TGF-β ligand trap polypeptide exposure-response analysis with select efficacy, biomarker, or safety measures. In certain embodiments, determining the at least one clinical endpoint comprises determining the incidence of anti-drug antibodies (ADAs) of TGF-β ligand trap. In various embodiments, the ADAs of the TGF-β ligand trap are directed to a TGF-β ligand trap polypeptide. In certain embodiments, determining the at least one clinical endpoint comprises determining summary measures of nivolumab trough observed serum concentration (Ctrough) and incidence of ADAs to nivolumab. In certain embodiments, determining the at least one clinical endpoint comprises summary measures of changes in corrected QT interval from baseline by dose and association measures of corrected QT interval changes with TGF-β ligand trap pharmacokinetic exposure. For example, the TGF-β ligand trap pharmacokinetic exposure is a TGF-β ligand trap polypeptide pharmacokinetic exposure. In certain embodiments, determing the at least one clinical endpoint comprises determining summary measures of pharmacokinetic parameters of TGF-β ligand trap metabolites. For example, the TGF-β ligand trap metabolites are TGF-β ligand trap polypeptide metabolites. In certain embodiments, determing the least one clinical endpoint comprises determining pharmokinetic parameter summary measures evala power model.

In certain embodiments of any of the methods described herein, the methods result in improvement of at least one symptom of the cancer, e.g., tumor size. In various embodiments, the methods comprise performing an efficacy assessment. In various embodiments, the efficacy assessment comprises an efficacy assessment of the antitumor activity of a TGF-β ligand trap (e.g., a TGF-β ligand trap polypeptide). For example, the efficacy assessment comprises making a tumor measurement, using for example RECIST v1.1, or imaging of a tumor. In certain embodiments, imaging may demonstrate tumor response or progression after treatment with the methods described. In certain embodiments, the tumor measurement or imaging of the tumor comprises contrast-enhanced computerized tomography (CT) of the chest, abdomen, pelvis, and all other known and/or suspected sites of disease is performed for tumor assessments. In certain embodiments, CT or magnetic resonance imaging (MRI) of the neck of the subject (e.g., a subject having SCCHN), is performed. In certain embodiments, the subject has a contraindication for CT intravenous contrast, and a non-contrast CT of the chest and a contrast-enhanced MRI of the neck (for SCCHN participants), abdomen, pelvis, and other known/suspected sites of disease is performed. In certain embodiments, the subject has a contraindication for both MRI and CT intravenous contrasts, and a non-contrast CT of the chest and a non-contrast MRI of the neck (for SCCHN participants), abdomen, pelvis, and other known/suspected sites of disease is performed. In certain embodiments, the subject has a contraindication for MRI (e.g., incompatible pacemaker) in addition to contraindication to CT intravenous contrast, and a non-contrast CT of the neck (for SCCHN participants), chest, abdomen, pelvis, and other known/suspected sites of disease is performed. In certain embodiments, the CT portion of the PET-CT is used for RECIST v1.1 measurements.

In certain embodiments of any of the methods provided herein, the occurrence of adverse events associated with the methods/treatments is less frequent than observed for existing standard therapies known to provide clinical benefit. In certain embodiments of any of the methods provided herein, the occurrence of adverse events associated with the methods/treatments is less frequent than observed for chemotherapy, radiation therapy, or other immunotherapies known to provide clinical benefit.

In certain embodiments of any of the methods provided herein, the methods/treatments are observed to be and/or are minimally immunogenic. In certain embodiments, the immunogenicity of the methods/treatments provided herein is determined by detection and characterization of antibodies to the TGF-β ligand trap (e.g., TGF-β ligand trap polypeptides) or nivolumab performed using a validated method. In certain embodiments, the antibodies may be further characterized and/or evaluated for their ability to neutralize the activity of the TGF-β ligand trap polypeptides or nivolumab.

In certain embodiments of any of the methods provided herein, PK, PK/PD, and exposure-response (ER) analyses for the TGF-β ligand trap polypeptides or nivolumab are conducted. In certain embodiments, the PK, PK/PD, and exposure-response (ER) measurements for treatements with the TGF-β ligand trap polypeptides and nivolumab in combination are improved compared to those observed with either treatment alone. The PK parameters assessed include, but are not limited to, maximum observed serum concentration (Cmax), time of maximum observed serum concentration (Tmax), area under the serum concentration-time curve from time zero extrapolated to time of last quantifiable concentration (AUC), Ctrough, serum concentration at the end of infusion (Ceoi), area under the concentration-time curve in 1 dosing interval (AUC(TAU)), area under the concentration-time curve from time zero extrapolated to infinite time (AUC(INF)).

Also provided herein is a kit for treating cancer in a subject in need thereof, wherein the kit comprises a treatment with a TGFβ trap (e.g., about 400 mg-1600 mg of any one of the polypeptides of SEQ ID NO: 1-5 and AVID200). For example, the treatment is a TGF-β ligand trap polypeptide monotherapy with a TGFβ trap as described herein. Also provided herein is a kit for treating cancer in a subject in need thereof, wherein the kit comprises a combination therapy, for example a nivolumab treatment (e.g., 360 mg or 480 mg) and a treatment with TGFβ trap described herein (e.g., about 400 mg to about 1600 mg). In various embodiments, the kit comprises instructions for use, for example instructions describing a method (e.g., a monotherapy with the TGFβ trap and a combination therapy with a TGFβ trap and nivolumab) as described herein. In various embodiments, the TGF-β ligand trap is formulated as a solution in a single-use vial. In various embodiments, the unit dose strength of the TGF-β ligand trap polypeptide in the formulation is 80 mg (8 mg/mL). In various embodiments, the nivolumab is formulated as a solution in a single use vial. In various embodiments, the unit dose strength of the nivolumab is 100 mg (10 mg/mL).

In certain embodiments of any of the methods provided herein, the subject has:
(a) elevated levels of a biomarker as compared to levels of the biomarker in a reference population; or
(b) decreased levels of a biomarker as compared to levels of the biomarker in a reference population; and
wherein the level of the biomarker is predictive of responsiveness to the nivolumab treatment and/or the treatment with the polypeptide. In certain embodiments, the elevated levels of the biomarker are about 10%, 20%, 25%, 30%, 40%, 50%, 60%, 70%, 75%, 80%, 90%, 100%, 200%, or 500% greater than the levels of the biomarker in the reference population. In certain embodiments, the elevated levels of the biomarker are equal to or about 10%, 20%, 25%, 30%, 40%, 50%, 60%, 70%, 75%, 80%, 90%, 100%, 200%, or 500% greater than the levels of the biomarker in the top 10%, top 5%, top 4%, top 3%, top 2%, or top 1% in the reference population. In certain embodiments, the decreased levels of the biomarker are about 10%, 20%, 25%, 30%, 40%, 50%, 60%, 70%, 75%, 80%, 90%, or 100% less than the levels of the biomarker in the reference population. In certain embodiments, the decreased levels of the biomarker are equal to or about 10%, 20%, 25%, 30%, 40%, 50%, 60%, 70%, 75%, 80%, 90%, or 100% less than the levels of the biomarker in the bottom 10%, bottom 5%, bottom 4%, bottom 3%, bottom 2%, or bottom 1% in the reference population.

In certain embodiments of any of the methods provided herein, the level of the biomarker is determined by:
(a) gene expression profiling for TGF-β epithelial-mesenchymal transition/cancer-associated fibroblasts (EMT/CAF), interferon gamma (IFNy) signatures, cluster of differentiation 8 (CD8) tumor infiltrating lymphocytes (TIL), and/or T/NK cells;
(b) monitoring TGF-β signaling pathways;
(c) cytokine profiling in tumors and/or periphery;
(d) peptide or protein profiling in a tissue;
(e) profiling circulating micro ribonucleic acid;
(f) profiling circulating deoxyribonucleic acid (ctDNA);
(g) whole exome sequencing; and/or
(h) biomarker immunostaining.

In certain embodiments, the level of the biomarker is the protein level of the biomarker. In certain embodiments, the level of the biomarker is the mRNA level of the biomarker. In certain embodiments, the mRNA level is determined by quantitative reverse transcription polymerase chain reaction (qRT-PCR). In certain embodiments, the level of the biomarker is in a tissue. For example, the level of the biomarker in the tissue is determined by tissue staining. In certain embodiments, the subject is a human. In certain embodiments, the biomarker is collagen. In certain embodiments, the biomarker is CD8 tumor infiltrating lymphocytes.

In certain embodiments of any of the methods provided herein, the reference population consists of 1, 5, 10, 25, 50, 75, 100, 200, 250, 300, 400, 500, or 1000 individuals. In certain embodiments, the reference population consists of healthy people. In certain embodiments, the reference population consists of people of the same age, weight, and/or gender as the subject. In certain embodiments, the reference population consists of people without cancer.

In certain embodiments of any of the methods provided herein, the method further comprises monitoring the level of the biomarker in the subject.

Also provided herein is use of a polypeptide comprising an amino acid sequence selected from any one of SEQ ID NO: 1 to 5, alone and/or in combination with nivolumab, in any of the methods of treating cancer provided herein.

Also provided herein is a polypeptide comprising an amino acid sequence selected from any one of SEQ ID NO: 1 to 5 for use, alone and/or in combination with nivolumab, in any of the methods of treating cancer provided herein.

### 3. BRIEF DESCRIPTION OF FIGURES

FIG. 1 shows relative IL-11 release for A549 cells post TGF-β pulse as a function of TGF-β concentration. A549 cells were treated with a fixed concentration of TGF-β (20 pM) in the absence or presence of serial dilutions of TGF-β ligand trap polypeptide. Cell supernatants were then collected and assayed for IL-11 release.

FIG. 2 shows dosing schedule for one- and six-month GLP toxicology studies for TGF-β ligand trap polypeptide.

FIG. 3 depicts the method/steps utilized in a TGF-β1 ELISA assay.

FIG. 4A shows a pharmacokinetic profile for a TGF-β ligand trap polypeptide. FIG. 4B shows sequestration of TGF-β1 from cynomolgus monkey serum (released by acid activation) following intraveneous administration of TGF-β ligand trap polypeptide.

FIG. 5A depicts the CD8 IHC stained images for samples administered only PBS. FIG. 5B depicts the CD8 IHC stained images for samples administered only TGF-β ligand trap polypeptide. FIG. 5C depicts the CD8 IHC stained images for samples administered only anti-PD-L1 antibody. FIG. 5D depicts the CD8 IHC stained images for samples administered both TGF-β ligand trap polypeptide and anti-PD-L1 antibody.

FIG. 6 shows the effect of combination TGF-β ligand trap polypeptide/anti-PD-L1 antibody therapy on tumor size in an in vivo color cancer model.

### 4. DETAILED DESCRIPTION

Provided herein are immunotherapies for treating a disease or disorder such as a hyperproliferative malignancy, e.g., a cancer. The immunotherapies provided herein include various methods and compositions. More specifically, provided herein are combination treatments with a TGF-β ligand trap (see Section 4.2) and an immune checkpoint inhibitor (see Section 4.3.1).

In certain embodiments, provided herein are methods and compositions for treating a disease or disorder such as a cancer using a TGF-β ligand trap, such as a polypeptide comprising an amino acid sequence selected from any one of the amino acid sequences of SEQ ID NO: 1 to 5 (see Table 1), or an amino acid sequence that is at least 95%, 96%, 97%, 98%, or 99% identical to any one of the amino acid sequences of SEQ ID NO: 1 to 5 or a fragment thereof, and an immune checkpoint inhibitor. In certain embodiments, provided herein are methods and compositions for treating a disease or disorder such as a cancer using a polypeptide comprising an amino acid sequence of SEQ ID NO: 3, or AVID200 TGF-β ligand trap. Such an immune checkpoint inhibitor can inhibit, decrease, or interfere with the activity of a negative checkpoint regulator. Combination methods and for using a TGF-β ligand trap and an immune checkpoint inhibitor provided herein are described in more detail in Section 4.3 below.

### 4.1 Definitions

Techniques and procedures described or referenced herein include those that are generally well understood and/or commonly employed using conventional methodology by those skilled in the art. Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “effective amount” or “therapeutically effective amount” as used herein refers to the amount of a therapeutic compound, a combination of therapeutic compounds or pharmaceutical compositions thereof provided herein, which is sufficient to result in the desired outcome.

The terms “subject” and “patient” may be used interchangeably. As used herein, in certain embodiments, a subject is a mammal. In specific embodiments, the subject is a human. In one embodiment, the subject is a mammal, e.g., a human diagnosed with or suffering from a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or disorder resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease.

The terms “prevent,” “preventing,” and “prevention” refer to reducing the likelihood of the onset (or recurrence) of a disease, disorder, condition, or associated symptom(s).

The terms “inhibition”, “inhibit”, “inhibiting” refer to a reduction in the activity or expression of a polypeptide or protein (e.g., TGFβ1and TGFβ3) or reduction or amelioration of a disease, disorder, or condition or a symptom thereof. Inhibiting as used here can include partially or totally blocking stimulation, decreasing, preventing, or delaying activation, or inactivating, desensitizing, or down-regulating protein or enzyme activity.

The term “immune checkpoint inhibitor” refers to a molecule that inhibits, decreases or interferes with the activity of a negative checkpoint regulator. In certain embodiments, immune checkpoint inhibitors for use with the methods and compositions disclosed herein can inhibit the activity of a negative checkpoint regulator directly, or decrease the expression of a negative checkpoint regulator, or interfere with the interaction of a negative checkpoint regulator and a binding partner (e.g., a ligand). Immune checkpoint inhibitors for use with the methods and compositions disclosed herein include a protein, a polypeptide, a peptide, an antisense oligonucleotide, an antibody, an antibody fragment, or an inhibitory RNA molecule that targets the expression of a negative checkpoint regulator.

A “negative checkpoint regulator” refers to a molecule that down-regulates immune responses (e.g., T-cell activation) by delivery of a negative signal to T-cells following their engagement by ligands or counter-receptors. Exemplary functions of a negative-checkpoint regulator are to prevent out-of-proportion immune activation, minimize collateral damage, and/or maintain peripheral self-tolerance. In certain embodiments, a negative checkpoint regulator is a ligand or receptor expressed by an antigen presenting cell. In certain embodiments, a negative checkpoint regulator is a ligand or receptor expressed by a T-cell. In certain embodiments, a negative checkpoint regulator is a ligand or receptor expressed by both an antigen presenting cell and a T-cell.

The term “antibody” also known as an immunoglobulin, as used herein, refers to a large (e.g., Y-shaped) protein that binds to an antigen and/or target. Antibodies are used by the immune system to identify and neutralize foreign objects such as bacteria and viruses. The antibody recognizes a unique part of the antigen, because each tip of the “Y” of the antibody contains a site that is specific to a site on an antigen, allowing these two structures to bind with precision. An antibody (e.g., a multi-chain antibody) may consist of four polypeptide chains, two heavy chains and two light chains connected by interchain cysteine disulfide bonds. For example, antibodies (e.g., multi-chain antibodies) include human IgG1 and human IgG4 which have four interchain disulfide bonds (e.g., two heavy chain-light chain interchain disulfide bonds and two hinge heavy chain-heavy chain interchain disulfide bonds), human IgG2 which has six interchain disulfide bonds (e.g., four heavy chain-light chain interchain disulfide bonds and two hinge heavy chain-heavy chain interchain disulfide bonds), and human IgG3 which has thirteen interchain disulfide bonds (e.g., eleven heavy chain-light chain interchain disulfide bonds and two hinge heavy chain-heavy chain interchain disulfide bonds).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, ADC function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody,” as used herein, refers to an antibody obtained from a population of substantially homogeneous antibodies, e.g., the individual antibodies comprising the population are identical except for possible mutations, e.g., naturally occurring mutations, that may be present in minor amounts.

The terms “about” and “approximately” mean within 20%, within 15%, within 10%, within 9%, within 8%, within 7%, within 6%, within 5%, within 4%, within 3%, within 2%, within 1%, or less of a given value or range. In the context of the dosing cycle regime, the term “about” means within 5 days, within 4 days, within 3 days, within 2 days, or within 1 day of the recited day.

As used in the present disclosure and claims, the singular forms “a”, “an” and “the” include plural forms unless the context clearly dictates otherwise.

The term “between” as used in a phrase as such “between A and B” or “between A-B” refers to a range including both A and B.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document.

### 4.2 Transforming Growth Factor-β (TGF-β) Ligand Traps

A Transforming Growth Factor-β (TGF-β) ligand trap that can be used with the methods and compositions disclosed herein can comprise one extracellular binding domain (ECD) of the TGF-β receptor. In certain embodiments, a TGF-β ligand trap can comprise more than one ECD of the TGF-β receptor such as the TGF-β ligand traps described in WO 2020/069372 (Elstar Therapeutics, Inc.) and US 2015/0225483 (Merck Patent GMBH). There are three known TGF-β receptor types, the TGF-β type I receptor is the signaling chain and does not bind the ligand; the TGF-β type II receptor binds ligands TGF-β1 and TGF-β3 with high affinity but only weakly binds TGF-β2; and the TGF-β type III receptor functions as a co-receptor. In certain embodiments, a TGF-β ligand trap that can be used with the methods and compositions disclosed herein comprises at least one ectodomain of a TGF-β type II receptor (TGF-βRII) (Accession number P37173).

Wherein a TGF-β ligand trap comprises more than one TGF-β ECD, the domains can be joined to each other by a peptide linker, for example a short peptide linker. In certain embodiments, the linker can comprise multiples glycine residues such as the linker described in WO 2008/157367 (Genzyme Corporation). Alternatively, in certain embodiments, the TGF-β ECDs can be joined together by way of a natural linker. In certain embodiments, the TGF-β binding domain(s) of a TGF-β ligand trap can be fused or linked to an additional protein or domain. The additional protein can be the constant region of an immunoglobulin as is disclosed in WO 1998/48024 (Biogen Inc); WO 2011/109789 (The Johns Hopkins University); WO 2015/077540 (The Brigham and Womens Hospital, Inc.); US 20200002425 (Altor Biosciences Corporation); and US 10,316,076 (Acceleron Pharma, Inc.). A Fc domain fused to a TGF-β ligand trap can derived from IgG1, IgG2, IgG3, or IgG4.

A TGF-β ligand trap that can be used with the methods and compositions disclosed herein. The TGF-β ligand trap is in certain embodiments a polypeptide. In various embodiments, the polypeptide comprises from N-terminus to C-terminus (i) an amino acid sequence consisting of amino acids I1 to D272 of any one of SEQ ID NO: 1-5; and (ii) the Fc region of an antibody heavy chain. In certain embodiments, the Fc region is the Fc region of an IgG1 antibody. In certain embodiments, the Fc region is the Fc region of an IgG2 antibody. In certain embodiments, the polypeptide further comprises a glycine rich linker fused to the N terminus of the antibody Fc portion of the sequence.

Exemplary TGF-β ligand traps that can be used with the methods and compositions disclosed herein are provided in Table 1. In certain embodiments, the TGF-β ligand trap is a polypeptide comprising an amino acid sequence selected from the amino acid sequence of SEQ ID NO:1 to 5. In some embodiments, the TGF-β ligand trap is a polypeptide comprising an amino acid sequence that is 95%, 96%, 97%, 98% or 99% identical to a sequence selected from SEQ ID NO:1 to 5. In some embodiments, the TGF-β ligand trap is a polypeptide comprising an amino acid sequence set forth in Table 1, or an amino acid sequence that is 95%, 96%, 97%, 98% or 99% identical to a sequence set forth in Table 1. In some embodiments, the TGF-β ligand trap is a polypeptide consisting of an amino acid sequence selected from the amino acid sequences of SEQ ID NO:1 to 5 (see, Table 1).

TABLE 1

Amino Acid Sequences of Exemplary TGF-β Ligand Trap Polypeptides

| SEQ ID NO: | Amino Acid Sequence |
|---|---|
| 1 | IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG |
| 2 | IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG |
| 3 | IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKP |
| | GETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG |
| 4 | IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG |
| 5 | IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGSGGGSGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG |

In certain embodiments, the TGF-β ligand trap is a polypeptide having the N-terminus of any one of SEQ ID: 1-5 precisely as shown in Table 1. In other embodiments, the TGF-β ligand trap is a polypeptide having the C-terminus of any one of SEQ ID: 1-5 precisely as shown in Table 1. In some embodiments, the TGF-β ligand trap is a polypeptide having the N-terminus and C terminus of any one of SEQ ID: 1-5 precisely as shown in Table 1.

It is understood in the art that polypeptides can be post-translationally modified in a variety of ways. Examples of post-translational modifications commonly observed include, but are not limited to, phosphorylation, glycosylation, sialylation, ubiquitination, nitrosylation, methylation, acetylation, lipidation, formation of disulfide bonds, and cross-linking of amino acids. In certain embodiments, a TGF-β ligand trap that can be used with the methods and compositions disclosed herein is a polypeptide comprising an amino acid sequence selected from the amino acid sequence of SEQ ID NO:1 to 5, or a sequence that is 85%, 86%, 87%, 88%, 89%, 90%, 91%, 92%, 93%, 94%, 95%, 96%, 97%, 98% or 99% identical to a sequence selected from SEQ ID NO: 1 to 5 as shown in Table 1, wherein the polypeptide further comprises one or more post-translational modifications.

A TGF-P ligand trap that can be used with the methods and compositions disclosed herein can be fused or linked to other immunomodulatory or targeting domains. These resulting polypeptides can be bifunctional such as those described in WO 2019241625 (Acceleron Pharma, Inc.); US 2015/0225483 (Merck Patent GMBH); or David et al., Oncoimmunology, 2017; or multifunctional such as US 20200140547 (The Johns Hopkins University); or WO 2020/069372 (Elstar Therapeutics, Inc.). A TGF-β ligand trap that can be used with the methods and compositions disclosed herein binds to TGF-β1 and TGF-β3 with low pM potency. In certain embodiments, a TGF-β ligand trap binds to TGF-β1 and TGF-β3 with a binding affinity (K_{D}) of 10 micromolar (µM), 5 µM, 1 µM, 500 nanomolar (nM), 100 nM, 50 nM, 10 nM, 1 nM, 100 picomolar (pM), 50 pM, 10 pM, 1 pM, or lower. In certain embodiments, the K_{D} of the TGF-P ligand trap against the TGF-β2 isoform is 1 nM, 5 nM, 10 nM, 50 nM, 100 nM, 500 nM, 1 µM, or higher. To determine the binding affinity for a TGF-β ligand trap disclosed herein, various assays can be used including, but are not limited to, Enzyme-Linked Immunosorbent Assays (ELISAs) and/or surface plasmon resonance (SPR) methods such as the Biacore system.

Additionally, a TGF-β ligand trap can inhibit TGF-β ligand binding to its receptor on cells resulting in the neutralization of the biological activity of TGF-β. In certain embodiments, the IC₅₀ of the TGF-β ligand trap against the TGF-β1 or TGF-β3 isoforms is 1 µM, 500 nM, 100 nM, 50 nM, 10 nM, 1 nM, 100 pM, 50 pM, 10 pM, 1 pM, or lower. Various assays can be used to determine the ability of the TGF-β ligand trap to neutralize TGF-β including, but not limited to, the in vitro bioassays described in WO 2008/157367 (Genzyme, Inc.). TGF-β isoforms can be determined using in vitro assays such as the Magnetic Luminex Performance Assay.

A TGF-β ligand trap that can be used with the methods and compositions disclosed herein can enhance the capacity of T-cells isolated from draining lymph nodes to specifically recognize and kill tumor cells. In certain embodiments, a TGF-β ligand trap will have high anti-tumor T-cell activating potency. In a specific embodiment, a TGF-β ligand trap that can be used with the methods and compositions disclosed herein has a higher anti-tumor T-cell-activating potency than that of the pan-neutralizing TGF-β antibody, 1D11 (Ling et al., 2013, PLoS One. 8(1): e54499; Watanabe H et al. 2020 Sci Rep 10 (1):9211). In certain embodiments, a TGF-β ligand trap that can be used with the methods and compositions described herein can be AVID200.

### 4.3 Combination Therapies

Provided herein are methods and compositions relating to the combination of a Transforming Growth Factor-β (TGF-β) ligand trap (e.g., a polypeptide comprising an amino acid sequence selected from any one of SEQ ID NO:1 to 5, and AVID200) and an immunotherapy. In certain embodiments, an immunotherapy comprises administering an immune checkpoint inhibitor. Immune checkpoint inhibitors, including agents that can be used as inhibitors of PD-1 mediated signaling such as inhibitors of PD-1 are set forth in Section 4.3.1.

### 4.3.1 Immune Checkpoint Inhibitors

The clinical use of immune-oncology agents targeting cytotoxic T-lymphocyte-associated protein 4 (CTLA-4) and the programmed cell death receptor-1 (PD-1) and its ligand PD-L1, have resulted in improvements over the standard of care in the treatment of many cancer types. While these checkpoint inhibitors have produced improved clinical responses in such certain cancers, durable clinical responses only occur in approximately 10-45% of patients. Moreover, a significant number of tumors are either resistant or become refractory.

The immune checkpoint inhibitor to be administered in combination with the TGF-β ligand trap (e.g., AVID200) can be any pharmaceutical agent that inhibits or blocks the activity of an inhibitory immune checkpoint molecule. In specific embodiments, the activity is binding to the natural binding partner of the immune checkpoint molecule. If the immune checkpoint molecule is a receptor, the activity can be ligand-binding activity. If the immune checkpoint molecule is a ligand, the activity can be receptor-binding activity.

In specific embodiments, the immune checkpoint inhibitor to be administered in combination with the TGF-β ligand trap (e.g., AVID200) is a negative checkpoint regulator that is involved in T-Cell activation (e.g., antagonist), or a positive or stimulatory checkpoint regulator that is involved in T-cell activation (e.g., agonist). In certain, more specific embodiments, such a negative checkpoint regulator, or positive or stimulatory checkpoint regulator is Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137, CGEN-15001T, CGEN-15022, CGEN-15027, CGEN-15049, CGEN-15052, or CGEN-15092. An overview such checkpoint regulators and drugs that target them is set forth in Table 2. In a specific embodiment, the immune checkpoint inhibitor is an inhibitor of PD-1, PD-L1, PD-L2, CTLA-4, LAG3, TIM-3, VISTA, A2AR, B7-H3, B7-H4, BTLA, IDO, or TDO.

TABLE 2

| | | | | | |
|---|---|---|---|---|---|
| Target for Immunotherapy | Mode of Action | Commercial Name | FDA Approval | Indication (human) | Preclinical Evaluation |
| PD-1 / PD1-L(1,2) (programmed cell death protein-1) | Antibodies that bind to PD-1 (Block receptor) | Nivolumab (Opdivo, Bristol Myers Squibb) | Yes | Metastatic Melanoma, Non-Small cell lung cancer | Yes. PD-1 Ab alone and in combination with CTLA4 have been systematically evaluated in 7 different mouse models. (Barnes et al, AACR Annual Meeting Poster # 3362 (2015)) |
| | | Pembrolizumab (Keytruda, MK-3475 Merck) | Yes | Metastatic Melanoma (Clinical trials for lung cancer, lymphoma, mesothelioma) | |
| | | Cemiplimab (Libtayo, Regeneron) | Yes | Cutaneous squamous cell carcinoma (CSCC) or locally advanced CSCC who are not | |
| | | | | candidates for curative surgery or curative radiation | P815-B7-H1-modified (Mastocytoma) have also been evaluated. (See Hirano et al., Cancer Res., 65(3), 1089-96 (2005)) |
| | | Pidilizumab (CT-011, Medivation) | No | Clinical Trials - multiple cancers | |
| | | Spartalizumab (PDR001, Novartis) | No | Solid tumors and lymphomas | |
| | | Camrelizumab (SHR1210, HengRui) | No | Relapsed or refractory classical Hodgkin lymphoma | |
| | | AMP-224 (AstraZeneca/MedI mmune and GlaxoSmithKline) | No | | |
| | | AMP-514 (MEDI0680, AstraZeneca) | No | | |
| | Antibodies bind PD-L1 (inhibit receptor binding) | Atezolizumab (Tecentriq, Roche) | Yes | Urothelial carcinoma and non-small cell lung cancer | |
| | | Avelumab (Bavencio, Merck Serono and Pfizer) | Yes | Metastatic merkel-cell carcinoma | |
| | | Durvalumab (Imfinzi, Astrazeneca) | Yes | Urothelial carcinoma and unresectable non-small cell lung cancer after chemoradiation | |
| | | BMS936559 (Bristol Myers Squibb) | No | Melanoma, Non-Small cell lung cancer, Ovarian Cancer Phase I: HIV | |
| CTLA4 (cytotoxic T lymphocyte-associated antigen 4) | By binding to CTLA4, the compounds enhance T-cell activation and block B7-1 and B7-2 T-cell co-stimulatory pathways | Ipilimumab (MDX010, Yervoy, Bristol Myers Squibb) | Yes | Melanoma | Yes. See Simpson et al. J.Exp.Med. 2013: 210(9):1695-710 variable abilities were allocated to different Ab clones. |
| | | Tremelimumab (CP-675,206, Pfizer) | No | Clinical trials - Multiple Cancers | |
| TIM-3 (T-Cell immunoglobulin and mucin-containing protein 3) | Anti-TIM-3 IgGs inhibit binding of TIM-3 to its receptor and/or Ligand (perhaps galectin-9) | LY3321367 (Eli Lilly) | No | n. a. | Yes. (See Sakuishi et al., J Exp. Med., 207(10), 2187-94 (2010)) CT26, 4T1 and B16 were tested in their respective background. |
| | | MBG453 (Novartis) | No | n. a. | |
| | | BMS-986258 (Bristol Myers Squibb) | No | n. a. | |
| LAG-3/ Galectin-3 (lymphocyte-activated gene-3) | Anti-LAG-3 antibodies / depletion of galectin-3 | relatlimab(BMS-986016, Bristol Myers Squibb) | Yes (Opdualag was approved in fixed-dose combination with nivolumab March 2022)) | Melanoma | Yes. NT2.5 (neu-expressing tumor cell line) reported in Kouo et al., Cancer Immunol Res. 3(4):412-23 (2015). B16 and MC38 reported in Woo et al., Cancer Res 72(4)_917-27 (.2012). |
| TIGIT (T-Cell immunoreceptor with Ig and ITIM domains) | Antibodies against TIGIT inhibit binding to PVR (receptor) disturbing the inhibitory signaling. Further CD226 can bind to PVR instead and deploy its T-Cell activating function/ Might be also involved in NK cell inhibition | Tiragolumab (MTIG7192A, Genentech/Roche) | No | Locally advanced or metastatic NSCLC | Yes. (See Johnston et al., Cancer Cell. 26(6):923-37 (2014)). |
| | | AB154 (Arcus Bioscience) | No | Advanced solid cancers | |
| | | vibostolumab (MK-7684, Merck) | No | Solid cancers | |
| | | BMS-986207 (Bristol Myers Squibb) | No | Advanced or metastatic solid cancers | |
| | | ASP8374 (Astellas) | No | Advanced or metastatic solid cancers | |

In certain embodiments, the immune checkpoint inhibitor can be an antibody, a small molecule, or an oligonucleotide (such as an aptamer, an shRNA, miRNA, siRNA, or antisense DNA). In specific embodiments, the immune checkpoint inhibitor has been approved by Food and Drug Administration (FDA) in the United States or a foreign counterpart agency for the treatment of the cancer or a disease caused by the pathogen.

In specific embodiments, the immune checkpoint inhibitor is an antibody that binds to and inhibits the activity of the immune checkpoint. Antibodies that can be the immune checkpoint inhibitor include, but are not limited to, monoclonal antibodies (including Fc-optimized monoclonal antibodies), polyclonal antibodies, multispecific antibodies (e.g., bispecific antibodies), antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments, and fusion proteins containing antibody fragments. In a specific embodiment, the antibody is a monoclonal antibody. In various embodiments, the antibody is a humanized antibody.

In certain embodiments, the immune checkpoint inhibitor is an inhibitor of PD-1. In a specific embodiment, the immune checkpoint inhibitor is a monoclonal antibody that binds to and inhibits the activity (e.g., ligand-binding activity) of PD-1.

In certain embodiments, the monoclonal antibody is selected from the group consisting of nivolumab, pidilizumab, MEDI0680, pembrolizumab, AMP-224, AMP-514, STI-A1110, TSR-042, AUR-012, cemiplimab, spartalizumab, camrelizumab, sintilimab, tislelizumab, and toripalimab.

In a specific embodiment, the monoclonal antibody is nivolumab, pidilizumab, MEDI0680, or pembrolizumab. In a further specific embodiment, the monoclonal antibody is nivolumab. In another specific embodiment, the immune checkpoint inhibitor that is an inhibitor of PD-1 is AMP-224. In another specific embodiment, the immune checkpoint inhibitor that is an inhibitor of PD-1 is pidilizumab. In another specific embodiment, the immune checkpoint inhibitor that is an inhibitor of PD-1 is pembrolizumab. In another specific embodiment, the immune checkpoint inhibitor that is an inhibitor of PD-1 is MEDI0680. In another specific embodiment, the immune checkpoint inhibitor that is an inhibitor of PD-1 is STI-A1110. In another specific embodiment, the immune checkpoint inhibitor that is an inhibitor of PD-1 is TSR-042. In another specific embodiment, the immune checkpoint inhibitor that is an inhibitor of PD-1 is AUR-012.

In certain embodiments, the immune checkpoint inhibitor is an inhibitor of PD-L1. In a specific embodiment, the immune checkpoint inhibitor is a monoclonal antibody that binds to and inhibits the activity (e.g., receptor-binding activity) of PD-L1.

In certain embodiments, the immune checkpoint inhibitor is selected from the group consisting of mpdl3280A, durvalumab, avelumab, BMS-936559, atezolizumab, RG7446, and STI-A1010.

In a specific embodiment, the monoclonal antibody is mpdl3280A, durvalumab, avelumab, BMS-936559, or atezolizumab. In another specific embodiment, the immune checkpoint inhibitor that is an inhibitor of PD-L1 is RG7446. In another specific embodiment, the immune checkpoint inhibitor that is an inhibitor of PD-L1 is STI-A1010.

In certain embodiments, the immune checkpoint inhibitor is an inhibitor of CTLA4 (for example, ipilimumab).

In certain embodiments, the immune checkpoint inhibitor is an inhibitor of LAG3 (for example, BMS-986016).

In certain embodiments, immune checkpoint inhibitors to be administered in combination with the TGF-β ligand trap (e.g., AVID200) include but are not limited to: OPDIVO® (nivolumab); YERVOY® (ipilimumab); relatilimab; linrodostat; EMPLICITI® (elotuzumab); BMS-986258; BMS 986315; BMS-986207; BMS-986249; and BMS-986218.

PD-1 inhibitors useful in the combinations described herein include any molecule capable of inhibiting, blocking, abrogating or interfering with the activity or expression of PD-1. In particular, an anti-PD-1inhibitor can be a small molecule compound, a nucleic acid, a polypeptide, an antibody, a peptibody, a diabody, a minibody, a single-domain antibody or nanobody, a single-chain variable fragment (ScFv), or a functional fragment or variant thereof. In one instance the PD-1 inhibitor is a small molecule compound (e.g., a compound having a molecule weight of less than about 1000 Da.) In other embodiments, useful PD-1 inhibitors in the combinations described herein include nucleic acids and polypeptides.

### 4.3.2 Nivolumab

In a specific embodiment, the anti-PD-1 antibody provided herein is nivolumab (Bristol Myers Squibb).

The heavy chain and light chain sequences of nivolumab are shown in Table 3.

TABLE 3

Heavy chain:
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE QEGNVFSCSV
LVKGFYPSDI AVEWESNGQP ENNYKTTPPV LDDDGSFFLY SRLTVDKSRW QEGNVFSCSV
MHEALHNHYT QKSLSLSLGK
(SEQ ID NO:6)

Light chain:
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
(SEQ ID NO:7)

The heavy chain variable region of nivolumab has an amino acid sequence of SEQ ID NO:9, comprising the CDR1 (SEQ ID NO: 23), CDR2 (SEQ ID NO:30) and CDR3 (SEQ ID NO:37) regions. The light chain variable region of nivolumab has an amino acid sequence of SEQ ID NO:16, comprising the CDR1 (SEQ ID NO:44), CDR2 (SEQ ID NO: 51) and CDR3 (SEQ ID NO:58) regions. The sequences of the heavy chain variable region, the light chain variable region, and CDR1, CDR2, and CDR3 regions of nivolumab are shown in Table 4 below.

TABLE 4

| V_{H} Sequence (SEQ ID NO:) | | | V_{L} Sequence (SEQ ID NO:) | | |
|---|---|---|---|---|---|
| QVQLVESGGG | WQPGRSLRL | DCKASGITFS | EIVLTQSPAT | LSLSPGERAT | LSCRASQSVS |
| NSGMHWVRQA | PGKGLEWVAV | IWYDGSKRYY | SYLAWYQQKP | GQAPRLLIYD | ASNRATGIPA |
| ADSVKGRFTI | SRDNSKNTLF | LQMNSLRAED | RFSGSGSGTD | FTLTISSLEP | EDFAVYYCQQ |
| TAVYYCATND | DYWGQGTLVT | VSS | SSNWPRTFGQ | GTKVEIK | |
| (SEQ ID NO:9) | | | (SEQ ID NO:16) | | |
| CDR1 (SEQ ID NO:) | CDR2 (SEQ ID NO:) | CDR3 (SEQ ID NO:) | CDR1 (SEQ ID NO:) | CDR2 (SEQ ID NO:) | CDR3 (SEQ ID NO:) |
| NSGMH | VIWYDGSKRYYADS VKG | NDDY | RASQSVSSYLA | DASNRAT | QQSSNWPRT |
| (SEQ ID NO:23) | (SEQ ID NO:30) | (SEQ ID NO:37) | (SEQ ID NO:44) | (SEQ ID NO:51) | (SEQ ID NO:58) |

### 4.3.3 Agonists and Stimulator Receptors

In some aspects, the treatment comprises administering an immune modulator that is an agent that binds and/or modulates, e.g., agonizes and/or increases, the activity of a checkpoint protein or receptor. In various embodiments, the immune modulator binds and/or modulates any one of OX40, CD27, CD28, CD40, and CD137. OX40, a member of tumor necrosis factor receptor superfamily, provides the critical second signal for T-cell activation, proliferation, survival, and especially memory cell differentiation. Croft et al., Immunol Rev, 229 (1) (2009), pp. 173-191. CD27, a 55 kDa type I transmembrane protein in the tumor necrosis factor receptor (TNFR) family, costimulates T-cell activation after binding to its ligand CD70. In humans, CD27 is constitutively and exclusively expressed by naive T cells and upregulated on activated T cells and B cells, while the expression of CD70, a type II transmembrane protein, is highly regulated and occurs only transiently on activated T cells, B cells, and dendritic cells (DCs) (Wajant, 2016). CD27 plays an important role in expansion of naive T cells, enhancement of effector function, and generation and long term maintenance of T cell immunity and the responder T cell pool (Hendricks et al., 2003 J Exp Med, 198, pp. 1369-1380). CD28 drives critical intracellular biochemical events including unique phosphorylation and transcriptional signaling, metabolism, and the production of key cytokines, chemokines, and survival signals that are essential for long-term expansion and differentiation of T cells. Bour-Jordan et al., Immunol Rev. 2011;241:180-205. D40, a transmembrane receptor of the tumor necrosis factor gene superfamily is expressed on a variety of cells, such as monocytes, B-cells, antigen presenting cells, endothelial, smooth muscle cells, and fibroblasts. The interaction between CD40 and CD40 ligand (CD40L) enhances the expression of cytokines, chemokines, matrix metalloproteinases, growth factors, and adhesion molecules, mainly through the stimulation of nuclear factor kappa B. Chatzigeorgiou et al., Biofactors. Nov-Dec 2009;35(6):474-83. CD137, or 4-1BB/tumor necrosis factor receptor superfamily (TNFRSF9) is expressed on CD4+ and CD8+ T cells rapidly after exposure to antigen and it has been shown that cross-linking of CD137, and the T cell receptor (TCR) on activated T cells can deliver co-stimulatory signals to T cells resulting in T cell proliferation, survival, memory formation and stronger effector function for cytotoxicity and cytokine production. Wortzman et al., Immunol. Rev. 2013;255:125-148.

### 4.3.4 Combination Therapies

Provided herein are methods of preventing and/or treating a diease or disorder (e.g., a cancer) in a subject comprising administering to a subject a TGF-β ligand trap (e.g any one of the polypeptides of SEQ ID NO: 1-5 and AVID200) and an immune checkpoint inhibitor described herein. In certain embodiments, provided herein are methods of preventing a diease or disorder (e.g., a cancer) in a subject comprising administering to a subject a TGF-β ligand trap (e.g., AVID200) and an immune checkpoint inhibitor (e.g., nivolumab) described herein. In certain embodiments, provided herein are methods of treating a diease or disorder (e.g., a cancer) in a subject comprising administering to a subject a TGF-β ligand trap (e.g., AVID200) and an immune checkpoint inhibitor described herein. In certain embodiments, a pharmaceutically effective amount of the TGF-β ligand trap (e.g., AVID200) is administered. In certain embodiments, the TGF-β ligand trap (e.g., AVID200) and the immune checkpoint inhibitor are concomitantly administered. In certain embodiments, the co-administration of the TGF-β ligand trap (e.g., AVID200) and the immune checkpoint inhibitor (e.g., nivolumab) is pharmaceutically effective to treat the disease or disorder (e.g., a cancer).

In certain embodiments, provided herein are methods of preventing and/or treating a diease or disorder (e.g., a cancer) in a subject comprising administering to a subject a TGF-β ligand trap (e.g., any one of the polypeptides of SEQ ID NO: 1-5 and AVID200) and an inhibitor of PD-1 mediated signaling described herein (e.g., an anti-PD-1 antibody). In certain embodiments, provided herein are methods of preventing a diease or disorder (e.g., a cancer) in a subject comprising administering to a subject a TGF-β ligand trap (e.g., AVID200) and an inhibitor of PD-1 mediated signaling described herein (e.g., an anti-PD-1 antibody). In certain embodiments, provided herein are methods of treating a diease or disorder (e.g., a cancer) in a subject comprising administering to a subject a TGF-β ligand trap (e.g., AVID200) and an inhibitor of PD-1 mediated signaling described herein (e.g., an anti-PD-1 antibody). In certain embodiments, a pharmaceutically effective amount of the TGF-β ligand trap (e.g., AVID200) is administered. In certain embodiments, TGF-β ligand trap (e.g., AVID200) and the inhibitor of PD-1 mediated signaling are concomitantly administered. In certain embodiments, the co-administration of TGF-β ligand trap (e.g., AVID200) and the inhibitor of PD-1 mediated signaling is pharmaceutically effective to treat the disease or disorder (e.g., a cancer).

In certain embodiments, the inhibitor of PD-1 mediated signaling is an anti-PD-1inhibitor.

In certain embodiments, provided herein are methods of preventing and/or treating a diease or disorder (e.g., a cancer) in a subject comprising administering to a subject a TGF-β ligand trap (e.g., any one of the polypeptides of SEQ ID NO: 1-5 and AVID200) and an anti-PD-1 antibody described herein (e.g., nivolumab). In certain embodiments, provided herein are methods of preventing a diease or disorder (e.g., a cancer) in a subject comprising administering to a subject a TGF-β ligand trap (e.g., any one of the polypeptides of SEQ ID NO: 1-5 and AVID200) and an anti-PD-1 antibody described herein (e.g., nivolumab). In certain embodiments, provided herein are methods of treating a diease or disorder (e.g., a cancer) in a subject comprising administering to a subject a TGF-β ligand trap (e.g., AVID200) and an anti-PD-1 antibody described herein (e.g., nivolumab). In certain embodiments, a pharmaceutically effective amount of the TGF-β ligand trap (e.g., AVID200) is administered. In certain embodiments, TGF-β ligand trap (e.g., AVID200) and the anti-PD-1 antibody are concomitantly administered. In certain embodiments, the co-administration of TGF-β ligand trap (e.g., AVID200) and the anti-PD-1 antibody described herein (e.g., nivolumab) is pharmaceutically effective to treat the disease or disorder (e.g., a cancer). In certain embodiments, the anti-PD-1 antibody is nivolumab.

In certain embodiments, provided herein are methods of preventing and/or treating a diease or disorder (e.g., a cancer) in a subject comprising administering to a subject AVID200 and nivolumab. In certain embodiments, provided herein are methods of preventing a diease or disorder (e.g., a cancer) in a subject comprising administering to a subject AVID200 and nivolumab. In certain embodiments, provided herein are methods of treating a diease or disorder (e.g., a cancer) in a subject comprising administering to a subject AVID200 and nivolumab. In certain embodiments, a pharmaceutically effective amount of AVID200 is administered. In certain embodiments, AVID200 and nivolumab are concomitantly administered. In certain embodiments, the co-administration of AVID200 and nivolumab is pharmaceutically effective to treat the disease or disorder (e.g., a cancer).

### Indications

Diseases and disorders that can be treated or prevented using the methods and compositions described herein include the oncology indications listed below. Specifically, these indications can be treated with the combination therapies of the TGF-β ligand trap (see Section 3.2) and an immunotherapy (see Section 4.3.1) as well as the monotherapy of the TGF-β ligand trap described in herein (see Section 4.4).

In some embodiments, the disease or disorder to be treated with the methods and compositions disclosed herein is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin’s disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin’s lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small- cell lung cancer, prostate cancer, small-cell lung cancer and spleen cancer.

In some embodiments, the disease or disorder is a hematological cancer, such as leukemia, lymphoma, or myeloma. In some embodiments, the cancer is selected from the group consisting of Hodgkin’s lymphoma, non-Hodgkin’s lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt’s leukemia (Burkitt’s lymphoma), acute biphenotypic leukemia, chronic myeloid lymphoma, chronic myelogenous leukemia (CML), and chronic monocytic leukemia. In a specific embodiment, the disease or disorder is myeloma. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the myeloma is multiple myeloma (MM).

In other embodiments, the disease or disorder is a solid tumor malignancy. In some embodiments, the solid tumor malignancy is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In some embodiments, the solid tumor malignancy is an advanced non-CNS-primary solid tumor. In some embodiments, the solid tumor malignancy is selected from a group consisting of gastric/gastroesophageal junction (GEJ) cancer, bladder/urothelial cancer, and non-small-cell lung cancer (NSCLC).

In certain embodiments, the disease or disorder is malignant solid tumor or myelofibrosis. In certain embodiments, the myelofibrosis is intermediate-2 or higher primary myelofibrosis (PMF), post-essential thrombocythemia or polycythemia-vera related MF (Post ET/PV MF). In other embodiments, the disease or disorder is advanced or metastatic malignancy.

In some embodiments, the disease or disorder is a cancer expressing TGF-β. In some embodiments, the disease or disorder is a cancer associated with high or elevated expression levels of TGF-β compared to a normal patient not having the cancer. Examples of such disease include, but are not limited to, pancreatic cancer, sarcomas, mesothelioma, cervical cancer, myelofibrosis, NSCLC, UC, colorectal cancer, SCCHN, hepatocellular carcinoma (HCC), ovarian cancer, and breast cancer.

In some embodiments, the subject is a human. In some embodiments, the subject is a subject diagnosed with cancer, e.g., a hematological malignancy.

### Dosing and Regimen

The amount of a prophylactic or therapeutic agent (the TGF-β ligand trap and the immune checkpoint inhibitor provided herein), or a composition provided herein that will be effective in the prevention and/or treatment of a disease or condition can be determined by standard clinical techniques. The precise dose to be employed in the formulation will also depend on the route of administration, and the seriousness of a disease or condition, and in some embodiments, should be decided according to the judgment of the practitioner and each patient’s circumstances.

The dose administered to a subject in the context of the present disclosure should be sufficient to affect a therapeutic response. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration.

The TGF-β ligand trap (e.g., any one of the polypeptides of SEQ ID NO: 1-5 and AVID200) and the immune checkpoint inhibitor can be formulated in different pharmaceutical compositions and administered separately to the subject in need thereof. Alternatively, the TGF-β ligand trap (e.g., AVID200) and the immune checkpoint inhibitor are administered together in the same pharmaceutical composition.

In some embodiments, the TGF-β ligand trap (e.g., any one of the polypeptides of SEQ ID NO: 1-5 and AVID200) and the immune checkpoint inhibitor are administered simultaneously. The term “simultaneously” means at the same time or within a short period of time, for example, less than 1 hour, less than 2 hours, less than 3 hours, less than 4 hours, or less than 12 hours.

In some embodiments, the TGF-β ligand trap (e.g., any one of the polypeptides of SEQ ID NO: 1-5 and AVID200) and the immune checkpoint inhibitor are not administered simultaneously, and instead the two compounds are administered at different times. In various embodiments, the TGF-β ligand (e.g., AVID200) trap is administered prior to the administration of the immune checkpoint inhibitor (e.g., nivolumab). Alternatively, the immune checkpoint inhibitor is administered prior to the administration of the the TGF-β ligand trap. In some embodiments, the TGF-β ligand trap (e.g., AVID200) and the immune checkpoint inhibitor are administered at least once during a dosing period. A dosing period as used herein is meant a period of time, during which each therapeutic agent has been administered at least once. A dosing cycle can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a dosing cycle is 1, 2, 3, 4, 5, 6, 7, 8, 9, or 10 weeks. In certain embodiments, a dosing period is a dosing cycle.

The prophylactic or therapeutic agent (the TGF-β ligand trap polypeptide (e.g., AVID200) and the immune checkpoint inhibitor provided herein) can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, MRI scan, or other commonly accepted evaluation modalities.

The prophylactic or therapeutic agent (the TGF-β ligand trap (e.g., AVID200) and the immune checkpoint inhibitor provided herein) can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of, e.g., at least 10 days. The term “intermittent” or “intermittently” as used herein is intended to mean stopping and starting at either regular or irregular intervals. For example, intermittent administration of the compound is administration for one to six days per week, administration in cycles (e.g., daily administration for two to eight consecutive weeks, then a rest period with no administration for up to one week), or administration on alternate days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks.

In certain embodiments, the prophylactic or therapeutic agent (the TGF-β ligand trap (e.g., any one of the polypeptides of SEQ ID NO: 1-5 and AVID200) and the immune checkpoint inhibitor provided herein) is administered once per day from one day to six months, from one week to three months, from one week to four weeks, from one week to three weeks, or from one week to two weeks.

In some embodiments, the TGF-β ligand trap (e.g., any one of the polypeptides of SEQ ID NO: 1-5 and AVID200) and the immune checkpoint inhibitor are administered for 1 to 10 cycles. In some embodiments, the TGF-β ligand trap (e.g., any one of the polypeptides of SEQ ID NO: 1-5 and AVID200) and the immune checkpoint inhibitor are administered for 1, 2, 3, 4, 5, 6, 7, 8, 9, or 10 cycles. In some embodiments, the TGF-β ligand trap (e.g., any one of the polypeptides of SEQ ID NO: 1-5 and AVID200) and the immune checkpoint inhibitor are administered for more than 10 cycles.

Regimens for administration of a combination described herein can be modified as necessary to include administration of the TGF-β ligand trap and/or the immune checkpoint inhibitor. Administration of such active agents, e.g., the TGF-β ligand trap and/or the immune checkpoint inhibitors, can be performed QD, QW, QM, BID, BIW, TIW, Q2W, Q3W, or Q4W, or in accordance with prescribing information for such immune checkpoint inhibitors as set forth, for example, in a package insert. In certain embodiments, the immune checkpoint inhibitor (e.g., nivolumab) is administered once every 1, 2, 3, or 4 weeks. In a specific embodiment, the immune checkpoint inhibitor (e.g., nivolumab) is administered once every week. In another specific embodiment, the immune checkpoint inhibitor (e.g., nivolumab) is administered once every 2 weeks. In another specific embodiment, the immune checkpoint inhibitor (e.g., nivolumab) is administered once every 3 weeks. In another specific embodiment, the immune checkpoint inhibitor (e.g., nivolumab) is administered once every 4 weeks. In certain embodiments, the TGF-β ligand trap (e.g., AVID200) is administered once every 1, 2, 3, or 4 weeks. In a specific embodiment, the TGF-β ligand trap (e.g., AVID200) is administered once every week. In a specific embodiment, the TGF-β ligand trap (e.g., AVID200) is administered once every 2 weeks. In another specific embodiment, the TGF-β ligand trap (e.g., AVID200) is administered once every 3 weeks. In another specific embodiment, the TGF-β ligand trap (e.g., AVID200) is administered once every 4 weeks.

In certain embodiments, the combination includes a TGF-β ligand trap (e.g., any one of the polypeptides of SEQ ID NO: 1-5 and AVID200) administered at an amount of greater than about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. In certain embodiments, the combination includes a TGF-β ligand trap (e.g., any one of the polypeptides of SEQ ID NO: 1-5 and AVID200) administered at an amount of greater than or equal to about: 200 mg, 250 mg, 300 mg, 350 mg, or 400 mg. For example, the TGF-β ligand trap is administered at a dose of about 400 mg, 800 mg, 1200 mg, or 1600 mg. In one embodiment, the combination described herein includes a TGF-β ligand trap (e.g., AVID200) administered at an amount greater than about: 100 mg/m², 200 mg/m², 300 mg/m², 400 mg/m², 500 mg/m², 600 mg/m², 700 mg/m², 800 mg/m², 900 mg/m², 1000 mg/m², 1500 mg/m², 2000 mg/m², 2500 mg/m², 3000 mg/m², 3500 mg/m², 4000 mg/m², 4500 mg/m², 5000 mg/m², 5500 mg/m², 6000 mg/m², 6500 mg/m², 7000 mg/m², 8000 mg/m², 9000 mg/m², or 10000 mg/m². In specific embodiments, the combination can include a TGF-β ligand trap (e.g., AVID200) administered at an amount of about 70 mg/m², about 180 mg/m², about 550 mg/m², or about 1100 mg/m².

In certain embodiments, the combination described herein includes an immune checkpoint inhibitor administered at an amount between 100 mg and 200 mg, between 200 mg and 300 mg, between 300 mg and 400 mg, between 400 mg and 500 mg, between 500 mg and 600 mg, between 600 mg and 700 mg, between 700 mg and 800 mg, between 800 mg and 900 mg, or between 900 mgand 1000 mg In specific embodiments, the combination described herein includes an immune checkpoint inhibitor (e.g., nivolumab) administered at an amount of about 240 mg, about 360 mg, about 400 mg, or about 480 mg. In a specific embodiment, the combination described herein includes an immune checkpoint inhibitor (e.g., nivolumab) administered at an amount of about 240 mg. In a specific embodiment, the combination described herein includes an immune checkpoint inhibitor (e.g., nivolumab) administered at an amount of about 480 mg.

In certain embodiments, the dosing of the immune checkpoint inhibitor in the combinations described herein is relative to the weight of the patient (i.e., mg/kg). In some embodiments, the immune checkpoint inhibitor is administered in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg.

In a specific embodiment, the immune checkpoint inhibitor (e.g., nivolumab) is administered at a dose of about 0.3 mg/kg, about 0.5 mg/kg, about 1.0 mg/kg, or about 3.0 mg/kg. In a specific embodiment, the immune checkpoint inhibitor (e.g., nivolumab) is administered at a dose of about 3.0 mg/kg. In a specific embodiment, the immune checkpoint inhibitor (e.g., nivolumab) is administered at a dose of about 4.5 mg/kg. For example, nivolumab is administered at a dose of about 4.5 mg/kg Q3W. In a specific embodiment, the immune checkpoint inhibitor (e.g., nivolumab) is administered at a dose of about 6.0 mg/kg. For example, embodiment, the immune checkpoint inhibitor (e.g., nivolumab) is administered at a dose of about 6 mg/kg Q4W.

In certain embodiments, the therapeutically effective amount of an anti-PD-1 antibody (e.g., nivolumab) is determined as an amount provided in a package insert provided with the PD-1 antibody (e.g., nivolumab). The term package insert refers to instructions customarily included in commercial packages of medicaments approved by the U.S. Food and Drug Administration (US FDA) or a similar regulatory agency of a country other than the USA (e.g., European Medicines Agency (EMA)), which contains information about, for example, the usage, dosage, administration, contraindications, and/or warnings concerning the use of such medicaments.

### Patient Population

The subjects treated in accordance with the methods described herein can be any mammals such as rodents and primates, and in a preferred embodiment, humans. In certain embodiments, the methods described herein can be used to treat cancer in any mammals such as rodents and primates, and in a preferred embodiment, in human subjects or human patients.

In certain embodiments, the subject treated in accordance with the methods described herein has elevated levels and/or activity of a biomarker as compared to levels of the biomarker in a reference population, wherein the level of the biomarker is predictive of responsiveness to the nivolumab treatment and/or the treatment with the polypeptide. In certain embodiments, the subject treated in accordance with the methods described herein has (i) cancer; and (ii) abnormal levels and/or activity of a biomarker as compared to levels of the biomarker in a reference population, wherein the level of the biomarker is predictive of responsiveness to the nivolumab treatment and/or the treatment with the polypeptide. In certain embodiments, the abnormal levels and/or activity of a biomarker are elevated as compared to levels of the biomarker in a reference population. In certain embodiments, the abnormal levels and/or activity of a biomarker are decreased as compared to levels of the biomarker in a reference population. In certain embodiments, the subject treated in accordance with the methods described herein has (i) cancer; and (ii) elevated levels and/or activity of a biomarker as compared to levels of the biomarker in a reference population, wherein the level of the biomarker is predictive of responsiveness to the nivolumab treatment and/or the treatment with the polypeptide. In certain embodiments, the elevated levels of the biomarker are about 10%, 20%, 25%, 30%, 40%, 50%, 60%, 70%, 75%, 80% 90%, 100%, 200%, or 500% greater than the levels of the biomarker in the reference population. In certain embodiments, the elevated levels of the biomarker are equal to or about 10%, 20%, 25%, 30%, 40%, 50%, 60%, 70%, 75%, 80%, 90%, 100%, 200%, or 500% greater than the levels of the biomarker in the top 10%, top 5%, top 4%, top 3%, top 2%, or top 1% in the reference population. In certain embodiments, the subject treated in accordance with the methods provided herein has a disease associated with elevated levels of the biomarker. In certain embodiments, the subject treated in accordance with the methods described herein has decreased levels of a biomarker as compared to levels of the biomarker in a reference population, wherein the level of the biomarker is predictive of responsiveness to the nivolumab treatment and/or the treatment with the polypeptide. In certain embodiments, the subject treated in accordance with the methods described herein has (i) cancer; and (ii) decreased levels and/or activity of a biomarker as compared to levels of the biomarker in a reference population, wherein the level of the biomarker is predictive of responsiveness to the nivolumab treatment and/or the treatment with the polypeptide. In certain embodiments, the decreased levels of the biomarker are about 10%, 20%, 25%, 30%, 40%, 50%, 60%, 70%, 75%, 80%, 90%, or 100% less than the levels of the biomarker in the reference population. In certain embodiments, 60%, 70%, 75%, 80%, 90%, or 100% less than the levels of the biomarker in the bottom 10%, bottom 5%, bottom 4%, bottom 3%, bottom 2%, or bottom 1% in the reference population. In certain embodiments, the subject treated in accordance with the methods provided herein has a disease associated with decreased levels of the biomarker. In certain embodiments, the disease and/or cancer is non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC), or pancreatic ductal adenocarcinoma (PDAC). In certain embodiments, the biomarker is collagen. In certain embodiments, the biomarker is CD8 tumor infiltrating lymphocytes.

In certain embodiments, data (e.g., biomarker levels or clinical symptoms) obtained from a reference population described herein is utilized to determine whether analogous data obtained from a subject treated or to be treated in accordance with the methods provided herein is pathologically high (e.g., increased) or low (e.g., decreased).

In certain embodiments, the size of the reference population can be 1, 5, 10, 25, 50, 75, 100, 200, 250, 300, 400, 500, or 1000 individuals. In certain embodiments, the reference population consists of random volunteers. In certain embodiments, the reference population consists of healthy people. In certain embodiments, the reference population consists of people of the same age, weight, and/or gender as the patient population. In certain embodiments, the reference population consists of people without cancer. In certain embodiments, the reference population refers to a subject treated in accordance with the methods provided herein prior to the onset of one or more symptoms of cancer.

In certain embodiments, the subject treated in accordance with the methods described herein has elevated levels and/or activity of a biomarker as compared to prior levels of the biomarker in the subject 1, 2, 3, 4, 5, 6, 8, 10, 12, 18, 24, or 48 months before the onset of symptoms or the diagnosis of cancer, wherein the level of the biomarker is predictive of responsiveness to the nivolumab treatment and/or the treatment with the polypeptide. In certain embodiments, the elevated levels of the biomarker are about 10%, 20%, 25%, 30%, 40%, 50%, 60%, 70%, 75%, 80%, 90%, 100%, 200%, or 500% greater than the prior levels of the biomarker in the subject 1, 2, 3, 4, 5, 6, 8, 10, 12, 18, 24, or 48 months before the onset of symptoms or the diagnosis of cancer. In certain embodiments, the subject treated in accordance with the methods provided herein has a disease associated with elevated levels of the biomarker. In certain embodiments, the subject treated in accordance with the methods described herein has decreased levels and/or activity of a biomarker as compared to prior levels of the biomarker in the subject 1, 2, 3, 4, 5, 6, 8, 10, 12, 18, 24, or 48 months before the onset of symptoms or the diagnosis of cancer, wherein the level of the biomarker is predictive of responsiveness to the nivolumab treatment and/or the treatment with the polypeptide. In certain embodiments, the decreased levels of the biomarker are about 10%, 20%, 25%, 30%, 40%, 50%, 60%, 70%, 75%, 80%, 90%, or 100% less than the prior levels of the biomarker in the subject 1, 2, 3, 4, 5, 6, 8, 10, 12, 18, 24, or 48 months before the onset of symptoms or the diagnosis of cancer. In certain embodiments, the subject treated in accordance with the methods provided herein has a disease associated with decreased levels of the biomarker. In certain embodiments, the biomarker is collagen. In certain embodiments, the biomarker is CD8 tumor infiltrating lymphocytes. In certain embodiments, the disease and/or cancer is non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC), or pancreatic ductal adenocarcinoma (PDAC).

As will be recognized by one of skill in the art, levels and/or activity of a biomarker can be compared independently and respectively to the level and/or activity of the biomarker in a corresponding reference population and/or prior levels in the subject. In certain embodiments, the level and/or activity of the biomarker is determined by: (a) gene expression profiling for TGF-β epithelial-mesenchymal transition/cancer-associated fibroblasts (EMT/CAF), interferon gamma (IFNγ) signatures, cluster of differentiation 8 (CD8) tumor infiltrating lymphocytes (TIL), and/or T/NK cells; (b) monitoring TGF-β signaling pathways; (c) cytokine profiling in tumors and/or periphery; (d) peptide or protein profiling in a tissue; (e) profiling circulating micro ribonucleic acid; (f) profiling circulating deoxyribonucleic acid (ctDNA); (g) whole exome sequencing; and/or (h) biomarker measurement and/or assessment. For example, the biomarker measurement and/or assessment comprises immunostaining a sample from the subject. In certain embodiments, the level of the biomarker is the protein level of the biomarker. In certain embodiments, the level of the biomarker is the mRNA level of the biomarker. In certain embodiments, the mRNA level is determined by quantitative reverse transcription polymerase chain reaction (qRT-PCR).

### 4.4 Monotherapy

In another aspect, provided herein are methods of treating a disease or disorder (e.g., a cancer) in a subject comprising administering to a subject a therapeutically effective amount of the TGFβ trap (e.g., any of polypeptides of the amino acid sequences of SEQ ID NO: 1-5 and AVID200), wherein the disease or disorder (e.g., a cancer expressing PD-1 and/or PDL1) is relapsed or refractory. In certain embodiments, the relapsed or refractory disease or disorder (e.g., a cancer) is resistant to treatment with an anti-PD-1 antibody (e.g., nivolumab). In certain embodiments, the subject was previously treated with an anti-PD-1 antibody (e.g., nivolumab).

In certain embodiments, the disease or disorder (e.g., a cancer expressing PD-1 and/or PDL1) is a disease or disorder (e.g., a cancer) disclosed in Section 4.3.4, treated by the combination therapy disclosed herein.

In certain embodiments, the relapsed or refractory disease or disorder (e.g., a cancer) is selected from the group consisting of non-small cell lung cancer (NSCLC), colorectal cancer, hepatocellular carcinoma, ovarian cancer, breast cancer, renal cell carcinoma (RCC) and pancreatic cancer.

In yet another aspect, provided herein are methods of treating relapsed or refractory non-small cell lung cancer (NSCLC) by administering a therapeutically effective amount of the TGFβ trap (e.g., any of polypeptides of the amino acid sequences of SEQ ID NO: 1-5 and AVID200), wherein the relapsed or refractory NSCLC is resistant to treatment with an anti-PD-1 antibody (e.g., nivolumab). In certain embodiments, the subject was previously treated with an anti-PD-1 antibody (e.g., nivolumab). In some embodiments, the relapsed or refractory NSCLC is Stage IIA or Stage IIB. The relapsed or refractory NSCLC can be a Stage IIIA or Stage IIIB cancer. The NSCLC can be a Stage IV cancer. Staging of cancers as described herein is described by the American Joint Committee on Cancer TNM classification of malignant tumours cancer staging notation as is well understood in the art.

In yet another aspect, provided herein are methods of treating relapsed or refractory colorectal cancer by administering a therapeutically effective amount of the TGFβ trap (e.g., any of polypeptides of the amino acid sequences of SEQ ID NO: 1-5 and AVID200), wherein the relapsed or refractory colorectal cancer is resistant to treatment with an anti-PD-1 antibody (e.g., nivolumab). In certain embodiments, the subject was previously treated with an anti-PD-1 antibody (e.g., nivolumab). In some embodiments the colorectal cancer is a Stage I cancer. In another embodiment, the colorectal cancer is a Stage IIA, Stage IIB, or Stage IIC cancer. In still another embodiment, the colorectal cancer is a Stage IIIA, Stage IIIB, or Stage IIIC cancer. In yet another embodiment, the colorectal cancer is a Stage IVA or Stage IVB cancer. In certain instances, the colorectal cancer is further characterized by the grade of the cancer. The colorectal cancer can be a Grade 1, Grade 2, Grade 3, or Grade 4 cancer in any of the stages provided herein.

In yet another aspect, provided herein are methods of treating relapsed or refractory hepatocellular carcinoma by administering a therapeutically effective amount of the TGFβ trap (e.g., any of polypeptides of the amino acid sequences of SEQ ID NO: 1-5 and AVID200), wherein the relapsed or refractory hepatocellular carcinoma is resistant to treatment with an anti-PD-1 antibody (e.g., nivolumab). In certain embodiments, the subject was previously treated with an anti-PD-1 antibody (e.g., nivolumab). In some embodiments, the relapsed or refractory hepatocellular carcinoma is Stage II cancer. The relapsed or refractory hepatocellular carcinoma can be a Stage IIIA, Stage IIIB, or Stage IIIC cancer. The hepatocellular carcinoma can be a a Stage IVA or Stage IVB cancer.

In yet another aspect, provided herein are methods of treating relapsed or refractory melanoma by administering a therapeutically effective amount of the TGFβ trap (e.g., any of polypeptides of the amino acid sequences of SEQ ID NO: 1-5 and AVID200), wherein the relapsed or refractory melanoma is resistant to treatment with an anti-PD-1 antibody (e.g., nivolumab). In certain embodiments, the subject was previously treated with an anti-PD-1 antibody (e.g., nivolumab). In some embodiments, the relapsed or refractory melanoma is a Stage IIA, IIB, or IIC cancer. The relapsed or refractory melanoma can be a Stage IIIA, Stage IIIB, or Stage IIIC cancer. The melanoma can be a Stage IV cancer.

In yet another aspect, provided herein are methods of treating relapsed or refractory ovarian cancer by administering a therapeutically effective amount of the TGFβ trap (e.g., any of polypeptides of the amino acid sequences of SEQ ID NO: 1-5 and AVID200), wherein the relapsed or refractory ovarian cancer is resistant to treatment with an anti-PD-1 antibody (e.g., nivolumab). In certain embodiments, the subject was previously treated with an anti-PD-1 antibody (e.g., nivolumab). In some embodiments the ovarian cancer is a Stage I cancer as defined by the FIGO Ovarian Cancer Staging standards. The ovarian cancer can be a Stage IA, IB, or IC (e.g., IC1, IC2, or IC3) cancer. In another embodiment, the ovarian cancer is a Stage II cancer. The ovarian cancer can be a Stage IIA or IIB cancer.

In yet another aspect, provided herein are methods of treating relapsed or refractory breast cancer by administering a therapeutically effective amount of the TGFβ trap (e.g., any of polypeptides of the amino acid sequences of SEQ ID NO: 1-5 and AVID200), wherein the relapsed or refractory breast cancer is resistant to treatment with an anti-PD-1 antibody (e.g., nivolumab). In certain embodiments, the subject was previously treated with an anti-PD-1 antibody (e.g., nivolumab). The breast cancer can be HER2 negative breast cancer. The breast cancer can be a HER2 positive breast cancer. The breast cancer can be triple-negative breast cancer. In some embodiments the breast cancer is a Stage IA or Stage IB cancer. In another embodiment, the breast cancer is a Stage IIA or Stage IIB cancer. In still another embodiment, the breast cancer is a Stage IIIA, Stage IIIB, or Stage IIIC cancer. In yet another embodiment, the breast cancer is a Stage IV cancer.

In yet another aspect, provided herein are methods of treating relapsed or refractory pancreatic cancer by administering a therapeutically effective amount of the TGFβ trap (e.g., any of polypeptides of the amino acid sequences of SEQ ID NO: 1-5 and AVID200), wherein the relapsed or refractory pancreatic cancer is resistant to treatment with an anti-PD-1 antibody (e.g., nivolumab). In certain embodiments, the subject was previously treated with an anti-PD-1 antibody (e.g., nivolumab). In some embodiments, the pancreatic cancer is locally advanced, surgically resected or unresected pancreatic cancer or metastatic pancreatic adenocarcinoma. In some embodiments the pancreatic cancer is a Stage IA or Stage IB cancer. In another embodiment, the pancreatic cancer is a Stage IIA or Stage IIB cancer. In still another embodiment, the pancreatic cancer is a Stage III cancer. In yet another embodiment, the pancreatic cancer is a Stage IV cancer.

### 4.5 Pharmaceutical Compositions

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds’ adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington’s Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The TGF-β ligand trap (e.g., TGF-β ligand trap polypeptide comprising the amino acid sequence of SEQ ID NOs: 1-5 or AVID200) can be formulated with one or more pharmaceutically acceptable excipient. Similarly, the immune checkpoint inhibitor can be formulated in a pharmaceutical composition with one or more pharmaceutically acceptable excipients. In some embodiments, provided herein is a combination therapy comprising a first pharmaceutical composition comprising a TGF-β ligand trap provided herein (e.g., TGF-β ligand trap polypeptide comprising the amino acid sequence of SEQ ID NOs: 1-5 or AVID200) and a first pharmaceutically acceptable excipient, and a second pharmaceutical composition comprising an immune checkpoint inhibitor (e.g., nivolumab) provided herein and a second pharmaceutically acceptable excipient. The first and the second pharmaceutically acceptable excipients can be the same or different. In some embodiments, a TGF-β ligand trap provided herein (e.g., TGF-β ligand trap polypeptide comprising the amino acid sequence of SEQ ID NOs: 1-5 or AVID200) and an immune checkpoint inhibitor provided herein are formulated together in a single pharmaceutical composition. In other embodiments, provided herein is a pharmaceutical composition comprising a TGF-β ligand trap provided herein (e.g., TGF-β ligand trap polypeptide comprising the amino acid sequence of SEQ ID NOs: 1-5 or AVID200), an immune checkpoint inhibitor provided herein, and one or more pharmaceutically acceptable excipient.

The TGF-β ligand traps and/or the immune checkpoint inhibitors provided herein can be formulated into suitable pharmaceutical compositions for different routes of administration, such as injection (subcutaneous, intramuscular, intravenous, intraperitoneal, intraosseous, intracardiac, intraarticular, and intracavernous), sublingual and buccal, rectal, vaginal, ocular, otic, nasal, inhalation, nebulization, cutaneous, or transdermal. The compounds described above may be formulated into pharmaceutical compositions using techniques and procedures well known in the art (see, e.g., Ansel, Introduction to Pharmaceutical Dosage Forms, (7th ed. 1999)).

In the compositions, effective concentrations of one or more compounds (i.e., the TGF-β ligand trap or the immune checkpoint inhibitor provided herein) or pharmaceutically acceptable salts are mixed with a suitable pharmaceutical excipient. In certain embodiments, the concentrations of the compounds in the compositions are effective for delivery of an amount, upon administration, that treats, prevents, or ameliorates one or more of the symptoms and/or progression of a disease or disorder provided herein (e.g., cancer, including solid cancer and blood borne cancer).

The active compound is in an amount sufficient to exert a therapeutically useful effect on the patient treated. The therapeutically effective concentration may be determined empirically by testing the compounds in in vitro and in vivo systems and then extrapolated therefrom for dosages for humans. The concentration of active compound in the pharmaceutical composition will depend on absorption, tissue distribution, inactivation, and excretion rates of the active compound, the physicochemical characteristics of the compound, the dosage schedule, and amount administered as well as other factors known to those of skill in the art.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The TGF-β ligand traps (e.g., any of polypeptides of the amino acid sequences of SEQ ID NO: 1-5 and AVID200) and/or the immune checkpoint inhibitors provided herein can be provided in pharmaceutical compositions in forms convenient to or facilitate their administration to a patient. For example, in some embodiments, when the immune checkpoint inhibitor is an anti-PD-1 antibody as described herein, the PD-1 inhibitor can be formulated as a ready to use solution for parenteral administration. In other embodiments, the PD-1 inhibitor, including for example an anti-PD-1 antibody, can be formulated as a powder (e.g., lyophilized powder) that can be resuspended in a liquid suitable for parenteral administration. In one embodiment, the PD-1 antibody is formulated for intravenous administration. In another embodiment, the TGF-β ligand trap and the PD-1 inhibitor are both formulated for intravenous administration. In another embodiment, the TGF-β ligand trap and the PD-1 inhibitor are both formulated for intravenous infusion administration.

Combinations described herein can be provided as controlled release pharmaceutical products, which have a goal of improving drug therapy over that achieved by their non controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects.

The combinations and pharmaceutical compositions described herein can be provided as part of a kit. Such kits can, for example, improve patient compliance or improve the accuracy or ease of preparation for administering the combination. The kit includes a TGF-β ligand trap as described herein. The kit also includes an anti-PD-1 inhibitor as described herein. The kit can include AMP-224. In some embodiments, the kit includes an anti-PD-1 antibody, as described herein, such as for example, nivolumab, pembrolizumab, pidilizumab, REGN2810, PDR 001, or MEDI0680. The kit can include a package insert or other information (e.g., prescribing information) useful for administration of the combination to a patient in need thereof, such as a cancer patient described herein.

Kits of the invention can include the combinations described herein (e.g., a TGF-β ligand trap and an anti-PD-1 antibody) having the same or different formulation. Each component of a combination described herein in a kit can be supplied in a separate, individual container. Alternatively or additionally, components of the combinations described herein can be supplied in a single container. In such instances, the container can be a container that is ready for administration to a patient in need thereof, such as for example, an IV bag, ampoule, or a syringe. In one embodiment, the PD-1 inhibitor can be supplied as, for example, a powder (e.g., lyophilized powder) or as a solution for parenteral administration. In certain embodiments, the PD-1 inhibitor is an anti-PD-1 antibody as described herein formulated for parenteral administration by, for example, intravenous administration.

The TGF-β ligand trap (e.g., any of polypeptides of the amino acid sequences of SEQ ID NO: 1-5 and AVID200) or the immune checkpoint inhibitor provided herein, or pharmaceutically acceptable salts thereof, may also be formulated to target a particular tissue, receptor, or other area of the body of the subject to be treated. Many such targeting methods are well known to those of skill in the art. All such targeting methods are contemplated herein for use in the instant compositions. In one embodiment, liposomal suspensions, including tissue-targeted liposomes, such as tumor-targeted liposomes, may also be suitable as pharmaceutically acceptable excipients. These may be prepared according to methods known to those skilled in the art.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

### 5. EXAMPLES

### 5.1 Example 1

### Protocol Summary - Study Overview

This is a study of TGF-β ligand trap polypeptides having an amino acid sequence selected from any one of SEQ ID: 1-5, administered as a single agent and in combination with nivolumab in participants with select advanced solid tumors: NSCLC, UC, HCC, MSS CRC, and PDAC. The TGF-β ligand trap polypeptides are evaluated in participants with advanced/metastatic NSCLC, UC, SCCHN, HCC, MSS CRC, or PDAC who are resistant or refractory to conventional therapies (including αPD-L1-based immunotherapy). The study is composed of 2 parts: Part 1A (dose escalation of TGF-β ligand trap polypeptide monotherapy), and Part 1B (dose ranging of TGF-β ligand trap polypeptide in combination with nivolumab). In each part, the TGF-β ligand trap polypeptide is evaluated on an every-3-weeks (Q3W) schedule of administration (with nivolumab, Q3W) and on an every-2-weeks (Q2W) schedule of administraton (with nivolumab, Q4W).

### Part 1A: Monotherapy

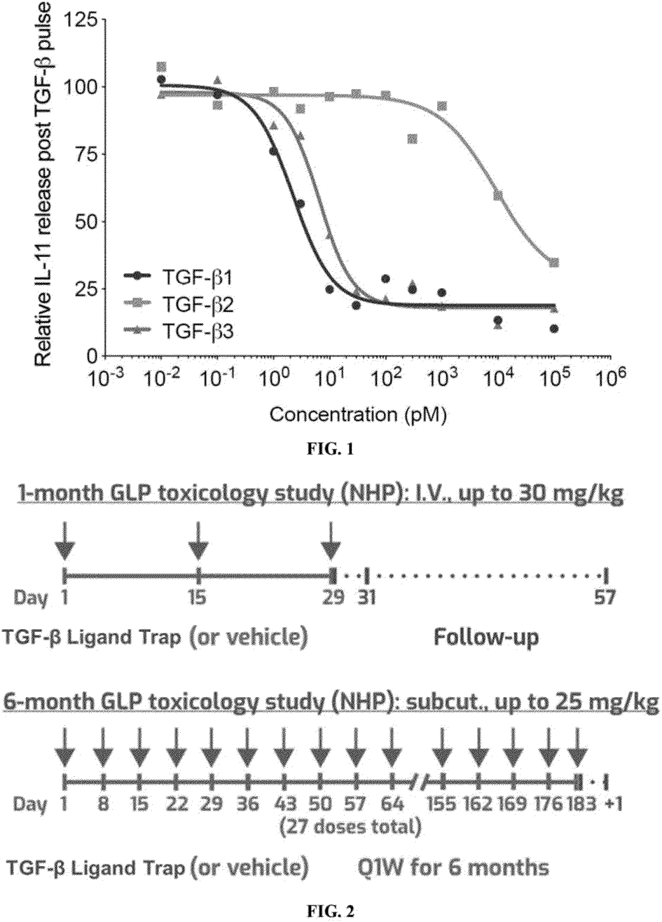

The monotherapy escalation portion of the study (Part 1A) evaluates increasing doses of TGF-β ligand trap polypeptides having an amino acid sequence selected from any one of SEQ ID: 1-5 in successive cohorts to determine the maximum tolerated dose (MTD)/ maximum administered dose (MAAD). The TGF-β ligand trap polypeptides of SEQ ID NO: 1-5 are comprised of isolated TGF-β receptors that were observed to inhibit the binding of TGF-β ligand to its cognate receptor on cells and inhibit in vivo tumor growth (WO2017/037634, Thwaites et al., Blood 130:2532 (2017) and FIGS. 1-5).

Part 1A evaluates the safety and tolerability of the TGF-β ligand trap polypeptides monotherapy based on dose-limiting toxicities (DLTs), using the time-to-event Bayesian optimal interval (TITE-BOIN) design to guide escalation decisions, and the overall assessment of available safety, PK, and PD data. Specifically, Part 1A evaluates 400 mg, 800 mg, and 1,600 mg doses of the TGF-β ligand trap polypeptides (flat doses, Q3W or Q2W). If a planned dose level is associated with an unacceptable safety profile, an intermediate dose level (by way of example, but not limited to, 1200 mg) is evaluated.

Participants continue treatment until disease progression, unacceptable toxicity, withdrawal of consent, completion of 35 cycles (Q3W) or 26 cycles (Q2W schedule, 4 week cycle length) of treatment (regardless of treatment delays), or the study ends.

### Part 1B: Combination Therapy

Part 1B evaluates the safety and tolerability over a range of doses of a TGF-β ligand trap polypeptide having an amino acid sequence selected from any one of SEQ ID: 1-5 in combination with nivolumab. Treatment in Part 1B is initiated in a staggered manner relative to Part 1A and evaluates 2 TGF-β ligand trap polypeptide doses that cleared for safety in Part 1A. Specifically, Part 1B evaluates 400 mg, 800 mg, and 1,600 mg doses of the TGF-β ligand trap polypeptides (flat doses, Q3W or Q2W). At no point does the dose of TGF-β ligand trap polypeptide administered in combination with nivolumab in Part 1B exceed the highest dose determined to be tolerable in Part 1A. In Part 1B, the dose of TGF-β ligand trap polypeptide is escalated, whereas the dose of nivolumab is fixed at 360 mg Q3W in the case of Q3W administration of the TGF-β ligand trap polypeptide and 480 mg Q4W in the case of Q2W administration of the TGF-β ligand trap polypeptide. However, if a planned dose level of TGF-β ligand trap polypeptide is associated with an unacceptable safety profile, an intermediate dose level (by way of example, but not limited to, 1200 mg) is evaluated, but the nivolumab dose will remain the same.

### Objectives and Endpoints

One objective of the study is to assess the safety and tolerability of TGF-β ligand trap polypeptides having an amino acid sequence selected from any one of SEQ ID: 1-5 as monotherapy and in combination with nivolumab in participants with advanced solid tumors. Another objective of the study is to assess the pharmacokinetic properties of intravenously delivered TGF-β ligand trap polypeptide as monotherapy and in combination with nivolumab. Another objective of the study is to assess the preliminary anticancer activity of said monotherapy and combination therapy by RECIST v1.1. Another objective of the study is to assess preliminary anticancer activity of monotherapy and combination therapy on progression-free and overall survival. Another objective of the study is to assess effects of TGF-β ligand trap polypeptides as monotherapy or in combination with nivolumab on TGF-β and other related signaling and signatures in tumor and peripheral samples. Another objective of the study is to explore potential relationships between TGF-β ligand trap polypeptide exposures, antitumor activity, select biomarkers, pharmacodynamic effects, and key safety measures. Another objective of the study is to explore the immunogenicity of intravenously administered TGF-β ligand trap polypeptides as monotherapy and in combination with nivolumab. Another objective of the study is to explore the pharmacokinetic properties and immunogenicity of nivolumab in combination with TGF-β ligand trap polypeptides. Another objective of the study is to assess the potential corrected QT interval pharmacokinetic-concentration and dose-related effect of TGF-β ligand trap polypeptides. Another objective of the study is to assess the pharmacokinetic properties of TGF-β ligand trap polypeptide metabolites. Another objective of the study is to assess TGF-β ligand trap polypeptide dose proportionality.

Clinical endpoints of the study include determining the incidence of adverse events, serious adverse events, adverse events meeting dose limiting toxicity criteria, adverse events leading to discontinuation, and death; protocol-defined maximum tolerated dose or maximum administered dose associated with monotherapy or combination therapy with a TGF-β ligand trap polypeptide having an amino acid sequence selected from any one of SEQ ID: 1-5. Another clinical endpoint of the study includes measurement of pharmacokinetic parameters of the TGF-β ligand trap polypeptides. Other clinical endpoints include determining objective response rate and duration of response associated with monotherapy or combination therapy. Still other clinical endpoints include determining overall survival, progression-free survival, or progression-free survival rate associated with monotherapy or combination therapy. Further clinical endpoints include evaluating tumor and peripheral biomarker changes between baseline and post-treatment samples from monotherapy and in combination therapy. Additional clinical endpoints include evaluating association measures from TGF-β ligand trap polypeptide exposure-response analysis with select efficacy, biomarker, or safety measures. Another clinical endpoint includes determining the incidence of ADAs of TGF-β ligand trap polypeptide. Another clinical endpoint includes determining summary measures of nivolumab Ctrough and incidence of nivolumab ADAs. Another clinical endpoint includes summary measures of changes in corrected QT interval from baseline by dose and association measures of corrected QT interval changes with TGF-β ligand trap polypeptide pharmacokinetic exposure. Another clinical endpoint includes determining summary measures of pharmacokinetic parameters of TGF-β ligand trap polypeptide metabolites. Another clinical endpoint includes determining pharmokinetic parameter summary measures evaluated using a power model.

### Study Design

The study includes a screening period of up to 28 days and begins when the participant signs the main informed consent form (ICF). Participants have a lesion that can be biopsied at an acceptable clinical risk as judged by the investigator in order to be eligible for the study. During the screening period, participants submit a fresh biopsy, which is evaluated for adequate and evaluable tumor tissue by the central laboratory

The screening period is followed by a treatment period. The treatment period comprises a dosing regimen of monotherapy or combination therapy with a TGF-β ligand trap polypeptide having an amino acid sequence selected from any one of SEQ ID: 1-5. The dosing regime of the TGF-β ligand trap polypeptide is Q3W or Q2W.

In the case of Q3W administration of the TGF-β ligand trap polypeptide, participants are treated for a maximum of up to 105 weeks (regardless of treatment delay), comprising of up to 35 treatment cycles (each cycle is 3 weeks in length).

Study visits in Part 1A (TGF-β ligand trap polypeptide Monotherapy Dose Escalation) are performed every week for the first 9 weeks following the first dose of study treatment. A TGF-β ligand trap polypeptide having an amino acid sequence selected from any one of SEQ ID: 1-5 is administered by IV every 3 weeks (Q3W). TGF-β ligand trap polypeptide infusions (60 to 90 minutes depending upon dose levels) included a 60-minute observation period after all infusions in Cycle 1 for each participant.

In Part 1B (TGF-β ligand trap polypeptide in Combination with Nivolumab Dose Ranging), nivolumab is administered by IV Q3W and is infused over 30 minutes. Nivolumab is given first, followed by a 30-minute observation period. TGF-β ligand trap polypeptide infusions include a 60-minute observation period after all infusions in Cycle 1 for each participant.

In the case of Q2W administration of the TGF-β ligand trap polypeptide, participants are treated for a maximum of up to 104 weeks (regardless of treatment delay), comprising of up to 26 treatment cycles (each cycle is 4 weeks in length). For the combination therapy, the TGF-β ligand trap polypeptide is given with nivolumab 480 mg administered by IV Q4W. The imaging frequency is Q8W (every 2 cycles) and the timing/frequency of other study visits/procedures is adjusted accordingly.

### Study Population

Participants have a histologically or cytologically confirmed locally advanced unresectable, metastatic, or recurrent select solid tumor are eligible to be included in the study. Eligible tumor types include non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC), and pancreatic ductal adenocarcinoma (PDAC).

Participants who are resistant/refractory to or intolerant of existing standard therapies known to provide clinical benefit are eligible for to be included in the study. In addition, Parparticipants with NSCLC, SCCHN, HCC, or UC must be resistant or refractory to anti-PD-L1-based immunotherapy.

Participants with controlled brain metastases are eligible. Controlled brain metastases are defined as no radiographic progression for at least 4 weeks following radiation and/or surgical treatment (or 4 weeks of observation if no intervention is clinically indicated), and no longer taking steroids for at least 2 weeks prior to first dose of study treatment, and with no new or progressive neurological signs and symptoms.

Participants who have a medical history of or current suffer from an uncontrolled or significant cardiovascular disease, including, but not limited to, aortic aneurysm, calcified/degenerative mitral or aortic valvulopathy, clinically significant arrhythmias, or myocarditis may be excluded. Participants who are known to have a connective tissue disease, such as Marfan, Ehlers-Danlos, or Loeys-Dietz syndrome, may also be excluded. Participants who have a medical requirement for chronic anticoagulant or antiplatelet agents (except low-dose aspirin) may also be excluded.

### Study Intervention

Study intervention is defined as any investigational intervention(s), marketed product(s), placebo or medical device intended to be administered to a study participant according to the study protocol.

Study InterventionsMonotherapy Dose Escalation. A TGF-β ligand trap polypeptide having an amino acid sequence selected from any one of SEQ ID: 1-5 is administered to participant. The TGF-β ligand trap polypeptide is formulated as a solution in a single-use vial. The unit dose strength of the TGF-β ligand trap polypeptide in the formulation is 80 mg (8 mg/mL). The TGF-β ligand trap polypeptide is administered at dosage levels of 400 mg, 800 mg, 1600 mg, and optionally an intermediary dosage level such as 1200 mg. The TGF-β ligand trap polypeptide is administered according to either a Q3W or Q2W dosing cycle schedule. The TGF-β ligand trap polypeptide is administered by IV infusion. The IV infusions are 60 to 90 minutes (depending upon dose levels) and include a 60-minute observation period after the infusion is given in Cycle 1 for each participant. Doses are given within three days of the scheduled day. For example, in the Q3W dosing cycles, participants may be dosed no less than 19 days from the previous dose.

Study Interventions-Combination Treatment with Nivolumab. A TGF-β ligand trap polypeptide having an amino acid sequence selected from any one of SEQ ID: 1-5 is administered to participant. The TGF-β ligand trap polypeptide is formulated as a solution in a single-use vial. The unit dose strength of the TGF-β ligand trap polypeptide in the formulation is 80 mg (8 mg/mL). The nivolumab is formulated as a solution in a single use vial. The unit dose strength of the nivolumab is 100 mg (10 mg/mL). The TGF-β ligand trap polypeptide is administered at dosage levels of 400 mg, 800 mg, 1600 mg, and optionally an intermediary dosage level such as 1200 mg. The TGF-β ligand trap polypeptide is administered according to either a Q3W or Q2W dosing cycle schedule. In the case of Q3W administration of the TGF-β ligand trap polypeptide, 360 mg of nivolumab is administered according to a Q3W dosing cycle schedule. In the case of Q2W administration of the TGF-β ligand trap polypeptide, 480 mg of nivolumab is administered according to a Q4W dosing cycle schedule. Both the TGF-β ligand trap polypeptide and the nivolumab are administered by IV infusion. A 30-minute infusion of nivolumab is followed by a 30-minute observation period, followed by a 60- to 90-minute infusion of the TGF-β ligand trap polypeptide (depending upon dose levels) and a 60-minute observation period after all infusions in Cycle 1 for each participant. Doses are given within three days of the scheduled day.

Dose Delay or Discontinuation. Dose delay criteria apply for all drug-related AEs. Participants who experience the following have all study intervention(s) withheld: Potential dose limiting toxicities (DLTs), until DLT relatedness is defined, Select AEs and laboratory abnormalities. For the purpose of guiding dose level decisions, DLTs will be defined based on the incidence, intensity, and duration of AEs, excluding toxicities clearly related to disease progression or intercurrent illness. Participants may resume treatment with study intervention(s) if, for example, they have completed AE management (e.g., corticosteroid taper) or are on ≤ 10 mg prednisone or equivalent.

### Study Assessments

Efficacy Assessments. Efficacy assessments for the antitumor activity of a TGF-β ligand trap polypeptide having an amino acid sequence selected from any one of SEQ ID: 1-5, alone and in combination with nivolumab, are based on tumor measurements, using RECIST v1.1. Screening images are acquired during the screening period. On-study images are acquired from the date of first dose until participant is off-study. Imaging may continue during follow-up from the study. Any imaging may demonstrate tumor response or progression. Contrast-enhanced CT of the chest, abdomen, pelvis, and all other known and/or suspected sites of disease is performed for tumor assessments. For participants with SCCHN, CT or MRI of the neck is also performed. If a participant has a contraindication for CT intravenous contrast, then a non-contrast CT of the chest and a contrast-enhanced MRI of the neck (for SCCHN participants), abdomen, pelvis, and other known/suspected sites of disease is obtained. If a participant has a contraindication for both MRI and CT intravenous contrasts, then a non-contrast CT of the chest and a non-contrast MRI of the neck (for SCCHN participants), abdomen, pelvis, and other known/suspected sites of disease is obtained. If a participant has a contraindication for MRI (eg, incompatible pacemaker) in addition to contraindication to CT intravenous contrast, then a non-contrast CT of the neck (for SCCHN participants), chest, abdomen, pelvis, and other known/suspected sites of disease is acceptable. If an imaging site can document that the CT performed as part of a PET-CT is of identical diagnostic quality to a diagnostic CT (with IV and oral contrast), then the CT portion of the PET-CT can be used for RECIST v1.1 measurements.

Adverse Events. Adverse events that are reported by participant such as, for example, those considered related to the study intervention or the study, are documented. The occurrence, frequency and cause of adverse events are assessed and evaluated.

Pharmacokinetics. Serum samples for the PK and immunogenicity assessments of the TGF-β ligand trap polypeptides having an amino acid sequence selected from any one of SEQ ID: 1-5 are collected for all participants receiving monotherapy and in combination with nivolumab. All concentration data is tabulated using summary statistics. These data may also be pooled for population PK, PK/PD, and exposure-response (ER) analyses. The PK parameters assessed include, but are not limited to, maximum observed serum concentration (Cmax), time of maximum observed serum concentration (Tmax), area under the serum concentration-time curve from time zero extrapolated to time of last quantifiable concentration (AUC), trough observed serum concentration (Ctrough), serum concentration at the end of infusion (Ceoi), area under the concentration-time curve in 1 dosing interval (AUC(TAU)), area under the concentration-time curve from time zero extrapolated to infinite time (AUC(INF)).

Immunogenicity Assessments. Antibodies to the TGF-β ligand trap polypeptides having an amino acid sequence selected from any one of SEQ ID: 1-5 and nivolumab (where relevant) are evaluated in serum samples collected from participants. The detection and characterization of antibodies to the TGF-β ligand trap polypeptides or nivolumab is performed using validated method(s). Samples collected for detection of antibodies to the TGF-β ligand trap polypeptides or nivolumab are also evaluated for TGF-β ligand trap polypeptides or nivolumab serum concentration to enable interpretation of the antibody data. Antibodies may be further characterized and/or evaluated for their ability to neutralize the activity of the TGF-β ligand trap polypeptides or nivolumab.

Biomarkers. Peripheral blood and tumor tissue samples are collected in this study at baseline, on-treatment, and upon disease progression (optional) to identify PD markers associated with response to the TGF-β ligand trap polypeptides having an amino acid sequence selected from any one of SEQ ID: 1-5 and nivolumab (where relevant). Biomarkers to be evaluated include, but are not limited to, gene expression profiling for TGF-β epithelial-mesenchymal transition/cancer-associated fibroblasts (EMT/CAF) and interferon gamma (IFNγ) signatures, cluster of differentiation 8 (CD8) tumor infiltrating lymphocytes (TIL), T/NK cells, effect on TGF-β signaling pathways, cytokine profiling in tumors and/or periphery and a peptide and/or a protein (e.g., collagen) profiling in tissues. Additional biomarkers, including, but not limited to, circulating micro ribonucleic acid, circulating deoxyribonucleic acid (ctDNA), and whole exome sequencing that are related to the mechanism of action and association with response in monotherapy and in combination therapy with nivolumab are evaluated.

### 5.2 Example 2

### Part A

Summary. This study demonstrated that TGF-β ligand trap polypeptides having an amino acid sequence of SEQ ID NO: 3 specifically neutralized TGF-β1 and TGF-β3 with picomolar potency.

Methods and Materials. TGF-β inhibition was monitored using an A549 cell-based IL-11 release functional assay (R&D Systems Human IL-11 Immunoassay Quantikine ELISA Kit, CAT#D1100). A period of time (24 hours) after cell seeding, A549 cells were treated with a fixed concentration of TGF-β (20 pM) in the absence or presence of serial dilutions of TGF-β ligand trap polypeptides having an amino acid sequence of SEQ ID NO: 3. Cell supernatants were collected and assayed for IL-11 by ELISA.

Results. The relative IL-11 release post TGF-β stimulation as a function of TGF-β trap concentration is shown in FIG. 1. From to this data, the IC50 values were determined to be ~3 pM for TGF-β1 and TGF-β3, with minimal inhibition of TGF-02 (~10 nM). Accordingly, the TGF-β ligand trap polypeptides having an amino acid sequence of SEQ ID NO: 3 are more than 1,500-fold more potent at neutralizing TGF-β1 and TGF-β3 than TGF-β2.

### Part B

Summary. This study demonstrated that the TGF-β ligand trap polypeptides having an amino acid sequence of SEQ ID NO: 3 were well-tolerated in non-human primates.

Methods and Materials. The safety of administering the TGF-β ligand trap polypeptide having an amino acid sequence of SEQ ID NO: 3 to non-human primates over one or six months was evaluated. Toxicology for administration over one month was evaluated by administering either the TGF-β ligand trap polypeptide or vehicle intravenously at doses up to 30 mg/kg on days 1, 15, and 29. Toxicology for administration over six months was evaluated by administering either the TGF-β ligand trap polypeptide or vehicle subcutaneously at doses up to 25 mg/kg on days on a Q1W schedule for six months. These dosing schedules are illustrated in FIG. 2.

Results. No related adverse effects were noted in one- and six-month repeat dosing GLP toxicology studies in non-human primates. The no-observed-adverse-effect level (NOAEL) was the highest dose tested.

### Part C

Summary. This study demonstrated that the TGF-β ligand trap polypeptide having an amino acid sequence selected of SEQ ID NO: 3 achieved prolonged systemic exposure and demonstrated peripheral target engagement across the entire dosing period in non-human primates.

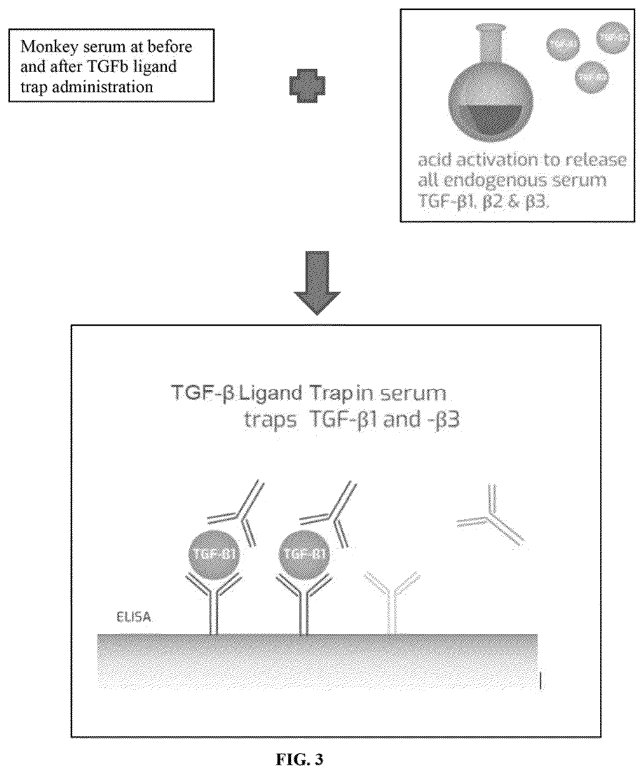

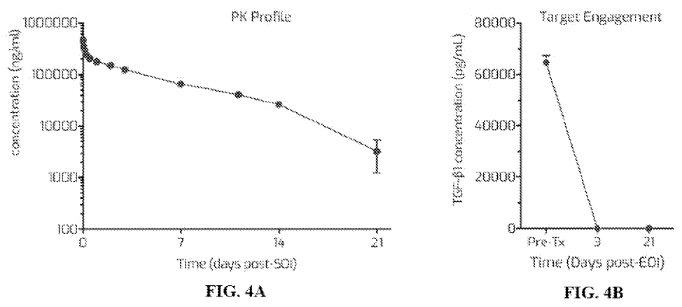

Methods and Materials. A single dose of the TGF-β ligand trap polypeptides having an amino acid sequence of SEQ ID NO: 3 was administered intravenously at 15 mg/kg in cynomolgus monkeys. Serum was collected at the timepoints post dosing shown in FIG. 4B. A TGF-β1 ELISA (R&D systems) was used to determine sequestration of all TGF-β1 contained in cynomolgus monkey serum (released by acid activation) following intravenous administration of the TGF-β ligand trap polypeptide. A diagram of the TGF-β1 ELISA assay is shown in FIG. 3.

Results. A pharmacokinetic profile of the TGF-β ligand trap polypeptide is shown in FIG. 4A. FIG. 4B shows sequestration of the TGF-β1 contained in cynomolgus monkey serum (released by acid activation) following intravenous administration of the TGF-β ligand trap polypeptide. This study demonstrated that the TGF-β ligand trap polypeptide having an amino acid sequence selected of SEQ ID NO: 3 achieved prolonged systemic exposure and demonstrated peripheral target engagement across the entire dosing period in non-human primates. Additional data (not shown) were obtained showing the TGF-β ligand trap polypeptide having an amino acid sequence selected of SEQ ID NO: 3 achieved dose proportional exposure in non-human primates.

### 5.3 Example 3

Summary. This study demonstrated that TGF-β ligand trap polypeptides having an amino acid sequence of SEQ ID NO: 3 in combination with anti-PD-L1 antibody enables infiltration of CD8+ T-Cells in TNBC model tumors.

Methods and Materials. EMT6 cells (1x10⁶) were orthotopically injected in the mammary fat pad of female Balb/c mice. Tumor size for treatment initiation was 100 mm³ on average (75-130 mm³) after 7 days. TGF-β ligand trap polypeptide (5 mg/kg) and anti-PD-L1 antibody (10 mg/kg) were administered twice weekly for 3 weeks by IP dose. Animals were sacrificed 14 days post treatment for histological analysis of the tumors. Tumor samples were formalin fixed, paraffin embedded and processed for CD8 IHC staining.

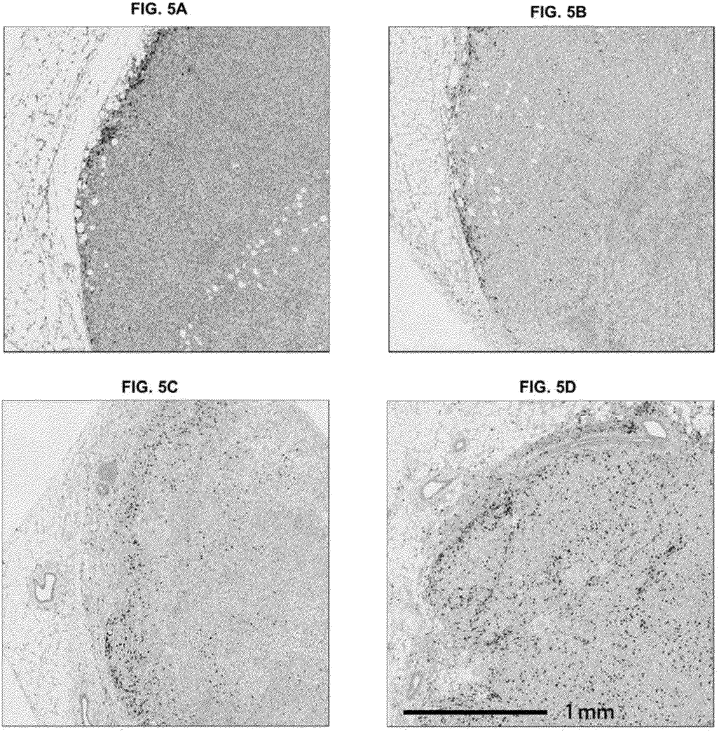

Results. FIG. 5 depicts the CD8 IHC stained images for samples administered/treated with only PBS (FIG. 5A), only TGF-β ligand trap polypeptide (FIG. 5B), only anti-PD-L1 (FIG. 5C) or both TGF-β ligand trap polypeptide and anti-PD-L1 (FIG. 5D). This study demonstrated that the TGF-β ligand trap polypeptides having an amino acid sequence of SEQ ID NO: 3 in combination with anti-PD-L1 enables infiltration of CD8+ T-Cells in TNBC model tumors.

### 5.4 Example 4

Summary. This study demonstrated that TGF-β ligand trap polypeptides having an amino acid sequence of SEQ ID NO: 3 sensitized tumors to immune checkpoint blockade in an in vivo colon cancer model.

Methods and Materials. A number (3x10⁵) MC-38 cells were injected subcutaneously into the right flank of 5-6-week-old female C57BL/6 mice. When the average tumor volume reached 50 mm³ - 100 mm³, the mice were randomized, and treatment was initiated (Day 1). Treatment of the subjects comprised administration of the TGF-β ligand trap polypeptide at a dose of 5 mg/kg twice per week and a dose of 200 µg anti-PD-L1 antibody three times per week for two weeks.

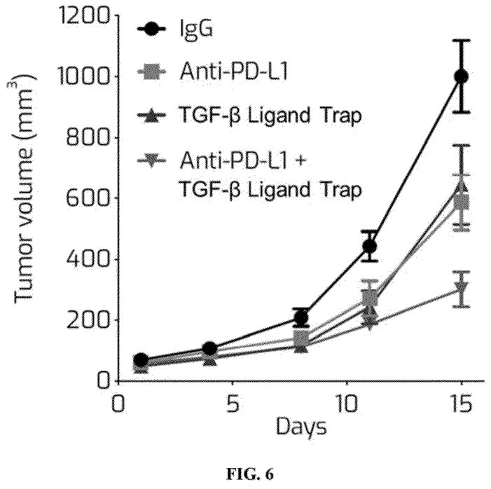

Results. FIG. 6 shows the growth inhibitory effect of the combination treatment of TGF-β ligand trap polypeptide and the anti-PD-L1 antibody therapy on tumor size.

### 5.5 Example 5

### Protocol Summary - Study Overview

As described in Example 1, a study was planned in which a TGF-β ligand trap polypeptide having the amino acid sequence of SEQ ID NO: 3 was administered as a single agent and in combination with nivolumab in participants with select advanced solid tumors: NSCLC, UC, HCC, MSS CRC, and PDAC. The TGF-β ligand trap polypeptide was evaluated in participants with advanced/metastatic NSCLC, UC, SCCHN, HCC, MSS CRC, or PDAC who are resistant or refractory to conventional therapies (including αPD-L1-based immunotherapy). The study was composed of 2 parts: Part 1A (dose escalation of TGF-β ligand trap polypeptide monotherapy), and Part 1B (dose ranging of TGF-β ligand trap polypeptide in combination with nivolumab). In each part, the TGF-β ligand trap polypeptide was evaluated on an every-3-weeks (Q3W) schedule of administration (with nivolumab, Q3W) and on an every-2-weeks (Q2W) schedule of administration (with nivolumab, Q4W).

### Part 1A: Monotherapy

The monotherapy escalation portion of the study (Part 1A) evaluated increasing doses of TGF-β ligand trap polypeptide having an amino acid sequence selected of SEQ ID NO: 3 in successive cohorts to determine the MTD/ MAAD.

Part 1A evaluated the safety and tolerability of the TGF-β ligand trap polypeptides monotherapy based on DLTs, using the TITE-BOIN design to guide escalation decisions, and the overall assessment of available safety, PK, and PD data. Specifically, Part 1A evaluated 400 mg, 800 mg, and 1,600 mg doses of the TGF-β ligand trap polypeptides (flat doses, Q3W or Q2W). If a planned dose level was associated with an unacceptable safety profile, an intermediate dose level (by way of example, but not limited to, 1200 mg) was evaluated.

Participants continue treatment until disease progression, unacceptable toxicity, withdrawal of consent, completion of 35 cycles (Q3W) or 26 cycles (Q2W schedule, 4-week cycle length) of treatment (regardless of treatment delays), or the study ends.

### Part 1B: Combination Therapy

Part 1B evaluated the safety and tolerability over a range of doses of a TGF-β ligand trap polypeptide having an amino acid sequence selected of SEQ ID NO: 3 in combination with nivolumab. Treatment in Part 1B was initiated in a staggered manner relative to Part 1A and evaluated 2 TGF-β ligand trap polypeptide doses that cleared for safety in Part 1A. Specifically, Part 1B evaluated 400 mg, 800 mg, and 1,600 mg doses of the TGF-β ligand trap polypeptides (flat doses, Q3W or Q2W). At no point did the dose of TGF-β ligand trap polypeptide administered in combination with nivolumab in Part 1B exceed the highest dose determined to be tolerable in Part 1A. In Part 1B, the dose of TGF-β ligand trap polypeptide was escalated, whereas the dose of nivolumab was fixed at 360 mg Q3W in the case of Q3W administration of the TGF-β ligand trap polypeptide and 480 mg Q4W in the case of Q2W administration of the TGF-β ligand trap polypeptide. However, if the planned dose level of TGF-β ligand trap polypeptides was associated with an unacceptable safety profile, an intermediate dose level (by way of example, but not limited to, 1200 mg) was evaluated, but the nivolumab dose remained the same.

### Objectives and Endpoints

One objective of the study was to assess the safety and tolerability of TGF-β ligand trap polypeptides having an amino acid sequence of SEQ ID NO: 3 as monotherapy and in a combination therapy with nivolumab in participants with advanced solid tumors. Another objective of the study was to assess the pharmacokinetic properties of intravenously delivered TGF-β ligand trap polypeptide as monotherapy and in combination with nivolumab. Another objective of the study was to assess the preliminary anticancer activity of said monotherapy and combination therapy by RECIST v1.1. Another objective of the study was to assess preliminary anticancer activity of monotherapy and combination therapy on progression-free and overall survival. Another objective of the study was to assess effects of TGF-β ligand trap polypeptides as monotherapy or in combination with nivolumab on TGF-β and other related signaling and signatures in tumor and peripheral samples. Another objective of the study was to explore potential relationships between TGF-β ligand trap polypeptide exposures, antitumor activity, select biomarkers, pharmacodynamic effects, and key safety measures. Another objective of the study was to explore the immunogenicity of intravenously administered TGF-β ligand trap polypeptides as monotherapy and in combination with nivolumab. Another objective of the study was to explore the pharmacokinetic properties and immunogenicity of nivolumab in combination with TGF-β ligand trap polypeptides. Another objective of the study was to assess the potential corrected QT interval pharmacokinetic-concentration and dose-related effect of TGF-β ligand trap polypeptides. Another objective of the study was to assess the pharmacokinetic properties of TGF-β ligand trap polypeptide metabolites. Another objective of the study was to assess TGF-β ligand trap polypeptide dose proportionality.

Clinical endpoints of the study included determining the incidence of adverse events, serious adverse events, adverse events meeting dose limiting toxicity criteria, adverse events leading to discontinuation, and death; protocol-defined maximum tolerated dose or maximum administered dose associated with monotherapy or combination therapy with a TGF-β ligand trap polypeptide having an amino acid sequence of SEQ ID No: 3. Another clinical endpoint of the study included measurement of pharmacokinetic parameters of the TGF-β ligand trap polypeptides. Other clinical endpoints included determining objective response rate and duration of response associated with monotherapy or combination therapy. Still other clinical endpoints included determining overall survival, progression-free survival, or progression-free survival rate associated with monotherapy or combination therapy. Further clinical endpoints included evaluating tumor and peripheral biomarker changes between baseline and post-treatment samples from monotherapy and in combination therapy. Additional clinical endpoints included evaluating association measures from TGF-β ligand trap polypeptide exposure-response analysis with select efficacy, biomarker, or safety measures. Another clinical endpoint included determining the incidence of ADAs of TGF-β ligand trap polypeptide. Another clinical endpoint included determining summary measures of nivolumab Ctrough and incidence of nivolumab ADAs. Another clinical endpoint included summary measures of changes in corrected QT interval from baseline by dose and association measures of corrected QT interval changes with TGF-β ligand trap polypeptide pharmacokinetic exposure. Another clinical endpoint included determining summary measures of pharmacokinetic parameters of TGF-β ligand trap polypeptide metabolites. Another clinical endpoint included determining pharmokinetic parameter summary measures evaluated using a power model.

### Study Design

The study included a screening period of up to 28 days and began when the participant signed the main ICF. Participants had a lesion that was biopsied at an acceptable clinical risk as judged by the investigator to be eligible for the study. During the screening period, participants submitted a fresh biopsy, which was evaluated for adequate and evaluable tumor tissue by the central laboratory

The screening period was followed by a treatment period. The treatment period comprised a dosing regimen of monotherapy or combination therapy with a TGF-β ligand trap polypeptide having an amino acid sequence of SEQ ID NO: 3. The dosing regimen of the TGF-β ligand trap polypeptide was Q3W or Q2W.

In the case of Q3W administration of the TGF-β ligand trap polypeptide, participants were treated for a maximum of up to 105 weeks (regardless of treatment delay), comprising of up to 35 treatment cycles (each cycle is 3 weeks in length).

Study visits in Part 1A (TGF-β ligand trap polypeptide Monotherapy Dose Escalation) were performed every week for the first 9 weeks following the first dose of study treatment. A TGF-β ligand trap polypeptide having an amino acid sequence of SEQ ID NO: 3 was administered by IV every 3 weeks (Q3W). TGF-β ligand trap polypeptide infusions (60 to 90 minutes depending upon dose levels) included a 60-minute observation period after all infusions in Cycle 1 for each participant.

In Part 1B (TGF-β ligand trap polypeptide in Combination with Nivolumab Dose Ranging), nivolumab was administered by IV Q3W and was infused over 30 minutes. Nivolumab was given first, followed by a 30-minute observation period. TGF-β ligand trap polypeptide infusions included a 60-minute observation period after all infusions in Cycle 1 for each participant.

In the case of Q2W administration of the TGF-β ligand trap polypeptide, participants were treated for a maximum of up to 104 weeks (regardless of treatment delay), comprising of up to 26 treatment cycles (each cycle is 4 weeks in length). For the combination therapy, the TGF-β ligand trap polypeptide was given with nivolumab 480 mg administered by IV Q4W. The imaging frequency was Q8W (every 2 cycles), and the timing/frequency of other study visits/procedures was adjusted accordingly.

### Study Population

Participants who had a histologically or cytologically confirmed locally advanced unresectable, metastatic, or recurrent select solid tumor were eligible to be included in the study. Eligible tumor types included NSCLC, UC, SCCHN, HCC, MSS CRC, and PDAC.

Participants who were resistant/refractory to or intolerant of existing standard therapies known to provide clinical benefit were eligible to be included in the study. In addition, Participants with NSCLC, SCCHN, HCC, or UC must have been resistant or refractory to anti-PD-L1-based immunotherapy.

Participants with controlled brain metastases were eligible. Controlled brain metastases was defined as no radiographic progression for at least 4 weeks following radiation and/or surgical treatment (or 4 weeks of observation if no intervention is clinically indicated), and no longer taking steroids for at least 2 weeks prior to first dose of study treatment, and with no new or progressive neurological signs and symptoms.

Participants who had a medical history of or current suffered from an uncontrolled or significant cardiovascular disease, including, but not limited to, aortic aneurysm, calcified/degenerative mitral or aortic valvulopathy, clinically significant arrhythmias, or myocarditis may have been excluded. Participants who were known to have a connective tissue disease, such as Marfan, Ehlers-Danlos, or Loeys-Dietz syndrome, may also have been excluded. Participants who had a medical requirement for chronic anticoagulant or antiplatelet agents (except low-dose aspirin) may also have been excluded.

### Study Intervention

Study intervention was defined as any investigational intervention(s), marketed product(s), placebo, or medical device intended to be administered to a study participant according to the study protocol.

Study Interventions-Monotherapy Dose Escalation. A TGF-β ligand trap polypeptide having an amino acid sequence of SEQ ID NO.: 3 was administered to participant. The TGF-β ligand trap polypeptide was formulated as a solution in a single-use vial. The unit dose strength of the TGF-β ligand trap polypeptide in the formulation was 80 mg (8 mg/mL). The TGF-β ligand trap polypeptide was administered at dosage levels of 400 mg, 800 mg, 1600 mg, and optionally an intermediary dosage level such as 1200 mg. The TGF-β ligand trap polypeptide was administered according to either a Q3W or Q2W dosing cycle schedule. The TGF-β ligand trap polypeptide was administered by IV infusion. The IV infusions were 60 to 90 minutes (depending upon dose levels) and included a 60-minute observation period after the infusion was given in Cycle 1 for each participant. Doses were given within three days of the scheduled day. For example, in the Q3W dosing cycles, participants may have been dosed no less than 19 days from the previous dose.

Study Interventions-Combination Treatment with Nivolumab. A TGF-β ligand trap polypeptide having an amino acid sequence of SEQ ID NO: 3 was administered to participant. The TGF-β ligand trap polypeptide was formulated as a solution in a single-use vial. The unit dose strength of the TGF-β ligand trap polypeptide in the formulation was 80 mg (8 mg/mL). The nivolumab was formulated as a solution in a single use vial. The unit dose strength of the nivolumab was 100 mg (10 mg/mL). The TGF-β ligand trap polypeptide was administered at dosage levels of 400 mg, 800 mg, 1600 mg, and optionally an intermediary dosage level such as 1200 mg. The TGF-β ligand trap polypeptide was administered according to either a Q3W or Q2W dosing cycle schedule. In the case of Q3W administration of the TGF-β ligand trap polypeptide, 360 mg of nivolumab was administered according to a Q3W dosing cycle schedule. In the case of Q2W administration of the TGF-β ligand trap polypeptide, 480 mg of nivolumab was administered according to a Q4W dosing cycle schedule. Both the TGF-β ligand trap polypeptide and the nivolumab were administered by IV infusion. A 30-minute infusion of nivolumab was followed by a 30-minute observation period, followed by a 60- to 90-minute infusion of the TGF-β ligand trap polypeptide (depending upon dose levels) and a 60-minute observation period after all infusions in Cycle 1 for each participant. Doses were given within three days of the scheduled day.

Dose Delay or Discontinuation. Dose delay criteria applied for all drug-related AEs. Participants who experienced the following had all study intervention(s) withheld: DLTs, until DLT relatedness was defined, Select AEs and laboratory abnormalities. For the purpose of guiding dose level decisions, DLTs were defined based on the incidence, intensity, and duration of AEs, excluding toxicities clearly related to disease progression or intercurrent illness. Participants resumed treatment with study intervention(s) if, for example, they completed AE management (e.g., corticosteroid taper) or were on ≤ 10 mg prednisone or equivalent.

### Study Assessments

Efficacy Assessments. Efficacy assessments for the antitumor activity of a TGF-β ligand trap polypeptide having an amino acid sequence of SEQ ID NO: 3, alone and in combination with nivolumab, were based on tumor measurements, using RECIST v1.1. Screening images were acquired during the screening period. On-study images were acquired from the date of first dose until participant was off-study. Imaging continued during follow-up from the study. Any imaging demonstrated tumor response or progression. Contrast-enhanced CT of the chest, abdomen, pelvis, and all other known and/or suspected sites of disease was performed for tumor assessments. For participants with SCCHN, CT or MRI of the neck was also performed. If a participant had a contraindication for CT intravenous contrast, then a non-contrast CT of the chest and a contrast-enhanced MRI of the neck (for SCCHN participants), abdomen, pelvis, and other known/suspected sites of disease was obtained. If a participant had a contraindication for both MRI and CT intravenous contrasts, then a non-contrast CT of the chest and a non-contrast MRI of the neck (for SCCHN participants), abdomen, pelvis, and other known/suspected sites of disease was obtained. If a participant had a contraindication for MRI (e.g., incompatible pacemaker) in addition to contraindication to CT intravenous contrast, then a non-contrast CT of the neck (for SCCHN participants), chest, abdomen, pelvis, and other known/suspected sites of disease was acceptable. If an imaging site could document that the CT performed as part of a PET-CT was of identical diagnostic quality to a diagnostic CT (with IV and oral contrast), then the CT portion of the PET-CT was used for RECIST v1.1 measurements.

Adverse Events. Adverse events that were reported by participant such as, for example, those considered related to the study intervention or the study, were documented. The occurrence, frequency and cause of adverse events were assessed and evaluated.

Pharmacokinetics. Serum samples for the PK and immunogenicity assessments of the TGF-β ligand trap polypeptide having an amino acid sequence of SEQ ID NO: 3 were collected for all participants receiving monotherapy and in combination with nivolumab. All concentration data were to be tabulated using summary statistics. These data were also pooled for population PK, PK/PD, and ER analyses. The PK parameters assessed include, but were not limited to, Cmax, Tmax, AUC, Ctrough, Ceoi, AUC(TAU), AUC(INF).

Immunogenicity Assessments. Antibodies to the TGF-β ligand trap polypeptide of SEQ ID NO: 3 and nivolumab (where relevant) were evaluated in serum samples collected from participants. The detection and characterization of antibodies to the TGF-β ligand trap polypeptide or nivolumab was performed using validated method(s). Samples collected for detection of antibodies to the TGF-β ligand trap polypeptide or nivolumab were also evaluated for TGF-β ligand trap polypeptide or nivolumab serum concentration to enable interpretation of the antibody data. Antibodies were further characterized and/or evaluated for their ability to neutralize the activity of the TGF-β ligand trap polypeptide or nivolumab.

Biomarkers. Peripheral blood and tumor tissue samples were collected in this study at baseline, on-treatment, and upon disease progression (optional) to identify PD markers associated with response to the TGF-β ligand trap polypeptide having an amino acid sequence of SEQ ID NO: 3 and nivolumab (where relevant). Biomarkers evaluated include, but were not limited to, gene expression profiling for TGF-β EMT/CAF and IFNy signatures, CD8 TIL, T/NK cells, effect on TGF-β signaling pathways, cytokine profiling in tumors and/or periphery and a peptide and/or a protein (e.g., collagen) profiling in tissues. Additional biomarkers included, but were not limited to, circulating micro ribonucleic acid, ctDNA, and whole exome sequencing that are related to the mechanism of action and association with response in monotherapy and in combination therapy with nivolumab are evaluated.

### RESULTS

In multiple in vitro experiments and preclinical in vivo models described above, the TGFβ-ligand trap described herein (SEQ ID NO: 3) was observed to bind TGFβ ligand 1 and TGFβ ligand 3, with low affinity for TGFβ ligand 2, and inhibit in vivo tumor growth. In some experiments the anti-tumor activity of the TGFB-ligand trap in combination with an anti-PD-L1 antibody was shown to be greater than that seen with either agent individually. The current clinical trial is the first-in-human analysis to investigate the optimized doses, regimen/schedule and the pharmacodynamic (PD) profile of a monotherapy treatment of the TGFB ligand trap of SEQ ID NO: 3 and the combination treatment with nivolumab to treat cancer. As of Jun. 14, 2022, Applicant had started recruiting patients in the above-described clinical trial. Samples were being collected and data (e.g., efficacy, safety, PD, and PK) obtained.

1. A method of treating cancer in a subject in need thereof, wherein the method comprises administering a nivolumab treatment and a treatment with a polypeptide comprising an amino acid sequence selected from any one of SEQ ID NO:1 to 5; wherein
(i) the nivolumab treatment comprises administering about 360-480 mg of nivolumab to the subject on about day 1 of a dosing cycle; and
(ii) the treatment with a polypeptide comprising an amino acid sequence selected from any one of SEQ ID NO:1 to 5 comprises administering a dose of about 400-1600 mg of the polypeptide to the subject on about day 1 of the dosing cycle.

2. The method of claim 1, wherein the nivolumab treatment comprises administering about 360 mg of nivolumab to the subject.

3. The method of claim 1 , wherein the nivolumab treatment comprises administering nivolumab to the subject a single time per dosing cycle.

4. The method of claim 1, wherein the treatment with a polypeptide comprising an amino acid sequence selected from any one of SEQ ID NO:1 to 5 comprises administering the polypeptide to the subject a single time per dosing cycle.

5. The method of claim 1, wherein the dosing cycle begins on day 1 and ends on day 21.

6. The method of any one of claims 1-5, wherein the dosing cycle is repeated about 10 to about 20 times, about 15 to about 25 times, about 20 to about 30 times, about 25 to about 35 times, or about 30 to about 40 times.

7. (canceled)

8. (canceled)

9. (canceled)

10. (canceled)

11. (canceled)

12. A method of treating cancer in a subject in need thereof, wherein the method comprises administering a nivolumab treatment and a treatment with a polypeptide comprising an amino acid sequence selected from any one of SEQ ID NO: 1 to 5; wherein
(i) the nivolumab treatment comprises administering about 360-480 mg of nivolumab to the subject on about day 1 of a dosing cycle; and
(ii) the treatment with a polypeptide comprising an amino acid sequence selected from any one of SEQ ID NO: 1 to 5 comprises administering a dose of about 400-1600 mg of the polypeptide to the subject on about day 1 of the dosing cycle and a dose of about 400-1600 mg of the polypeptide on about day 15 of the dosing cycle.

13. The method of claim 12, wherein the dose of the polypeptide that is administered on about day 1 and the dose of the polypeptide that is administered on about day 15 are the same.

14. The method of claim 12 , wherein the nivolumab treatment comprises administering about 480 mg of nivolumab to the subject.

15. The method of claim 12, wherein the nivolumab treatment consists of administering nivolumab to the subject once per dosing cycle.

16. The method of claim 12, wherein the treatment with a polypeptide comprising an amino acid sequence selected from any one of SEQ ID NO: 1 to 5 consists of administering the polypeptide to the subject twice per dosing cycle.

17. The method of claim 12, wherein the dosing cycle begins on day 1 and ends on day 28.

18. The method of any one of claims 12-17, wherein the dosing cycle is repeated about 10 to about 20 times, about 15 to about 25 times, about 20 to about 30 times, about 25 to about 35 times, or about 30 to about 40 times.

19. (canceled)

20. (canceled)

21. (canceled)

22. (canceled)

23. The method of claim 12, wherein the dosing cycle is repeated about 26 times.

24. The method of claim 1, wherein the dose of the polypeptide comprising an amino acid sequence selected from any one of SEQ ID NO:1 to 5 is about 400 mg, about 800 mg, about 1200 mg, or about 1600 mg.

25. (canceled)

26. (canceled)

27. (canceled)

28. The method of claim 1, wherein the nivolumab is present in a pharmaceutical composition that further comprises an excipient.

29. The method of claim 1, wherein the polypeptide comprising an amino acid sequence selected from any one of SEQ ID NO:1 to 5 is present in a pharmaceutical composition that further comprises an excipient.

30. The method of claim 1, wherein the nivolumab and the polypeptide comprising an amino acid sequence selected from any one of SEQ ID NO:1 to 5 are formulated together in a single pharmaceutical composition that further comprises an excipient.

31. The method of claim 1, wherein the polypeptide comprises an amino acid sequence consisting of SEQ ID 1.

32. The method of claim 1, wherein the polypeptide comprises an amino acid sequence consisting of SEQ ID 2.

33. The method of claim 1, wherein the polypeptide comprises an amino acid sequence consisting of SEQ ID 3.

34. The method of claim 1, wherein the polypeptide comprises an amino acid sequence consisting of SEQ ID 4.

35. The method of claim 1, wherein the polypeptide comprises an amino acid sequence consisting of SEQ ID 5.

36. The method of claim 1, wherein the cancer comprises an advanced solid tumor.

37. The method of claim 1, wherein the cancer is selected from the group consisting of non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC) and pancreatic ductal adenocarcinoma (PDAC).

38. The method of claim 1, wherein
(a) the method results in a reduction in the size of a tumor or in the number of tumors associated with the cancer; and/or
(b) the subject is resistant or refractory to or intolerant of existing standard cancer therapies known to provide clinical benefit; and/or
(c) the subject is resistant or refractory to immunotherapy that inhibits PD-1 signaling; and/or
(d) the subject is resistant or refractory to anti-PD-(L)1-based immunotherapy.

39. (canceled)

40. (canceled)

41. (canceled)

42. A method of preventing or treating a cancer in a subject comprising administering to a subject in need thereof an effective amount of a TGF-β ligand trap and nivolumab.

43. The method of claim 42, wherein the cancer is resistant or refractory to anti-PD1 based immunotherapy or anti-PD-L1-based immunotherapy.

44. The method of claim 43, wherein the anti-PD-1 based immunotherapy comprises an anti-PD-1 antibody.

45. The method of claim 44, wherein the anti-PD-1 antibody is nivolumab.

46. The method of claim 42, wherein the nivolumab is present in a pharmaceutical composition that further comprises an excipient.

47. The method of claim 42, wherein the TGF-β ligand trap is present in a pharmaceutical composition that further comprises an excipient.

48. The method of claim 42, wherein the nivolumab and the TGF-β ligand trap are formulated together in a single pharmaceutical composition that further comprises an excipient.

49. The method of claim 42, wherein the TGF-β ligand trap is a polypeptide comprising an amino acid sequence of any one of the sequences of SEQ ID Nos: 1 to 5.

50. (canceled)

51. (canceled)

52. (canceled)

53. (canceled)

54. (canceled)

55. The method of claim 42, wherein the TGF-β ligand trap is AVID200.

56. The method of claim 42, wherein the method comprises administering a therapeutically effective amount of the TGF-β ligand trap to the subj ect.

57. The method of claim 56, wherein the therapeutically effective amount is about 400 mg to about 1600 mg of the TGF-β ligand trap to the subject.

58. The method of claim 57, wherein the therapeutically effective amount comprises at least one dose selected from the group consisting of: about 400 mg, about 800 mg, about 1200 mg, and about 1600 mg.

59. (canceled)

60. (canceled)

61. (canceled)

62. (canceled)

63. The method of claim 42, wherein the method comprises administering the TGF-β ligand trap every two weeks (Q2W).

64. The method of claim 42, wherein the method comprises administering the TGF-β ligand trap every three weeks (Q3W).

65. The method of claim 42, wherein the TGF-β ligand trap is administered by intravenous infusion.

66. The method of claim 42, wherein nivolumab is administered at a dose of 360 mg or 480 mg.

67. (canceled)

68. The method of claim 42, wherein nivolumab is administered every three weeks Q3W or every four weeks (Q4W).

69. (canceled)

70. The method of claim 42, wherein nivolumab is administered by intravenous infusion.

71. The method of claim 42, wherein the cancer is relapsed or refractory.

72. The method of claim 71, wherein the cancer is relapsed or refractory to chemotherapy, radiation therapy, or immunotherapy.

73. The method of claim 72, wherein the relapsed or refractory cancer is resistant to treatment with nivolumab.

74. The method of claim 42, wherein the cancer is selected from the group consisting of non-small cell lung cancer (NSCLC), urothelial carcinoma (UC), colorectal cancer, squamous cell carcinoma of the head and neck (SCCHN), hepatocellular carcinoma (HCC), ovarian cancer, breast cancer, and pancreatic cancer.

75. The method of claim 42, wherein the method results in a reduction in the size of a tumor or in the number of tumors associated with the cancer.

76. The method of claim 42, wherein the method results in a reduction in an indicia of the presence or progression of the cancer.

77. The method of claim 1 further comprising administering at least one therapeutic agent.

78. The method of claim 77, wherein the at least one therapeutic agent comprises a therapeutic antibody.

79. The method of claim 1, wherein the cancer is associated with high levels of TGF-β.

80. The method of claim 1, wherein the subject has:
(a) elevated levels of a biomarker as compared to levels of the biomarker in a reference population; or
(b) decreased levels of a biomarker as compared to levels of the biomarker in a reference population; and
wherein the level of the biomarker is predictive of responsiveness to the nivolumab treatment and/or the treatment with the polypeptide.

81. The method of claim 80, wherein
(a) the elevated levels of the biomarker are about 10%, 20%, 25%, 30%, 40%, 50%, 60%, 70%, 75%, 80%, 90%, 100%, 200%, or 500% greater than the levels of the biomarker in the reference population; or
(b) the elevated levels of the biomarker are equal to or about 10%, 20%, 25%, 30%, 40%, 50%, 60%, 70%, 75%, 80%, 90%, 100%, 200%, or 500% greater than the levels of the biomarker in the top 10%, top 5%, top 4%, top 3%, top 2%, or top 1% in the reference population; or
(c) the decreased levels of the biomarker are about 10%, 20%, 25%, 30%, 40%, 50%, 60%, 70%, 75%, 80%, 90%, or 100% less than the levels of the biomarker in the reference population; or
(d) the decreased levels of the biomarker are equal to or about 10%, 20%, 25%, 30%, 40%, 50%, 60%, 70%, 75%, 80%, 90%, or 100% less than the levels of the biomarker in the bottom 10%, bottom 5%, bottom 4%, bottom 3%, bottom 2%, or bottom 1% in the reference population.

82. (canceled)

83. (canceled)

84. (canceled)

85. The method of claim 80, wherein the level of the biomarker is determined by:
(a) gene expression profiling for TGF-β epithelial-mesenchymal transition/cancer-associated fibroblasts (EMT/CAF), interferon gamma (IFNγ) signatures, cluster of differentiation 8 (CD8) tumor infiltrating lymphocytes (TIL), and/or T/NK cells;
(b) monitoring TGF-β signaling pathways;
(c) cytokine profiling in tumors and/or periphery;
(d) peptide or protein profiling in a tissue;
(e) profiling circulating micro ribonucleic acid;
(f) profiling circulating deoxyribonucleic acid (ctDNA);
(g) whole exome sequencing; and/or
(h) biomarker immunostaining.

86. The method of claim 80, wherein the level of the biomarker is the protein level of the biomarker.

87. The method of claim 80, wherein the level of the biomarker is the mRNA level of the biomarker.

88. The method of claim 87, wherein the mRNA level is determined by quantitative reverse transcription polymerase chain reaction (qRT-PCR).

89. The method of claim 80, wherein the level of the biomarker is in a tissue.

90. The method of any one of claim 80, wherein the subject is a human.

91. The method of any one of claim 80, wherein the biomarker is collagen.

92. The method of any one of claim 80, wherein the biomarker is CD8 tumor infiltrating lymphocytes.

93. The method of claim 80, wherein
(a) the reference population consists of 1, 5, 10, 25, 50, 75, 100, 200, 250, 300, 400, 500, or 1000 individuals; and/or
(b) the reference population consists of healthy people; and/or
(c) the reference population consists of people of the same age, weight, and/or gender as the subject; and/or
(d) the reference population consists of people without cancer.

94. (canceled)

95. (canceled)

96. (canceled)

97. The method of claim 1, wherein the method further comprises monitoring the level of the biomarker in the subject.

98. A method of treating cancer in a subject in need thereof, wherein the method comprises administering to the subject a polypeptide comprising an amino acid sequence selected from any one of SEQ ID NO: 1 to 5 at a dose of about 400-1600 mg.

99. The method of claim 98, wherein the method further comprises administering about 360 mg Q3W of nivolumab to the subject.

100. The method of claim 98, wherein the method further comprises administering about 480 mg Q4W of nivolumab to the subject.

101. The method of claim 98, wherein the treatment with a polypeptide comprising an amino acid sequence selected from any one of SEQ ID NO: 1 to 5 comprises administering the polypeptide to the subject a single time per dosing cycle.

102. The method of claim 98, wherein the dosing cycle begins on day 1 and ends on day 21.

103. The method of claim 98, wherein the dosing cycle is repeated about 10 to about 20 times, about 15 to about 25 times, about 20 to about 30 times, about 25 to about 35 times, or about 30 to about 40 times.

104. (canceled)

105. (canceled)

106. (canceled)

107. (canceled)

108. (canceled)

109. The method of claim 98, wherein the dose of the polypeptide comprising an amino acid sequence selected from any one of SEQ ID NO:1 to 5 is about 400 mg, about 800 mg, about 1200 mg, or about 1600 mg.

110. (canceled)

111. (canceled)

112. (canceled)

113. The method of claim 98, wherein the polypeptide is administered Q3W or Q4W.

114. (canceled)

115. The method of claim 98, wherein the nivolumab and the polypeptide comprising an amino acid sequence selected from any one of SEQ ID NO:1 to 5 are formulated together in a single pharmaceutical composition that further comprises an excipient.

116. (canceled)

117. (canceled)

118. (canceled)

119. (canceled)

120. (canceled)

121. The method of claim 98, wherein the cancer comprises an advanced solid tumor.

122. The method of claim 98, wherein the cancer is selected from the group consisting of non-small cell lung cancer (NSCLC), urothelial carcinoma (UC), squamous cell carcinoma of the head and neck (SCCHN), hepatocellular carcinoma (HCC), microsatellite-stable colorectal carcinoma (MSS CRC), renal cell carcinoma (RCC), and pancreatic ductal adenocarcinoma (PDAC).

123. The method of claim 98, wherein the method results in a reduction in the size of a tumor or in the number of tumors associated with the cancer.

124. The method of claim 98, wherein
(a) the subject is resistant or refractory to or intolerant of existing standard cancer therapies known to provide clinical benefit; and/or
(b) the subject is resistant or refractory to immunotherapy that inhibits PD-1 signaling; and/or
(c) the subject is resistant or refractory to anti-PD-(L)1-based immunotherapy.

125. (canceled)

126. (canceled)

127. (canceled)

128. (canceled)


SEQUENCE LISTING (58 sequences)

SEQ ID NO: 1 - protein, 499 aa, Artificial Sequence
  TGF-beta ligand trap Construct 1
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 2 - protein, 494 aa, Artificial Sequence
  TGF-beta ligand trap Construct 2
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 3 - protein, 496 aa, Artificial Sequence
  TGF-beta ligand trap Construct 3
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 504 aa, Artificial Sequence
  TGF-beta ligand trap Construct 4
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 507 aa, Artificial Sequence
  TGF-beta ligand trap Construct 5
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGSGGGSGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 440 aa, Artificial Sequence
  Nivolumab Heavy Chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 214 aa, Artificial Sequence
  Nivolumab Light Chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - skipped

SEQ ID NO: 9 - protein, 113 aa, Artificial Sequence [sequence omitted]

SEQ ID NO: 10-15 - skipped (6 entries)

SEQ ID NO: 16 - protein, 107 aa, Artificial Sequence [sequence omitted]

SEQ ID NO: 17-22 - skipped (6 entries)

SEQ ID NO: 23 - protein, 5 aa, Artificial Sequence
  Nivolumab heavy chain CDR1
NSGMH

SEQ ID NO: 24-29 - skipped (6 entries)

SEQ ID NO: 30 - protein, 17 aa, Artificial Sequence
  Nivolumab heavy chain CDR2
VIWYDGSKRYYADSVKG

SEQ ID NO: 31-36 - skipped (6 entries)

SEQ ID NO: 37 - protein, 4 aa, Artificial Sequence
  Nivolumab heavy chain CDR3
NDDY

SEQ ID NO: 38-43 - skipped (6 entries)

SEQ ID NO: 44 - protein, 11 aa, Artificial Sequence
  Nivolumab light chain CDR1
RASQSVSSYLA

SEQ ID NO: 45-50 - skipped (6 entries)

SEQ ID NO: 51 - protein, 7 aa, Artificial Sequence
  Nivolumab light chain CDR2
DASNRAT

SEQ ID NO: 52-57 - skipped (6 entries)

SEQ ID NO: 58 - protein, 9 aa, Artificial Sequence
  Nivolumab light chain CDR3
QQSSNWPRT